(19) United States
(12) Patent Application Publication
(10) Pub. No.: US 2021/0361767 A1
(43) Pub. Date: Nov. 25, 2021

(54) RESCUING CANCER PATIENTS FROM RESISTANCE TO TREATMENT WITH INHIBITORS OF PD-1/PD-L1 INTERACTIONS

(71) Applicant: Mayo Foundation for Medical Education and Research, Rochester, MN (US)

(72) Inventors: Haidong Dong, Rochester, MN (US); Jacob J. Orme, Rochester, MN (US)

(21) Appl. No.: 16/970,114

(22) PCT Filed: Feb. 14, 2019
(86) PCT No.: PCT/US2019/018107

§ 371 Date: Aug. 14, 2020

Related U.S. Application Data
(60) Provisional application No. 62/664,748, filed on Apr. 30, 2018.
(60) Provisional application No. 62/630,530, filed on Feb. 14, 2018.

(51) Int. Cl. A61K 39/395; A61K 31/496; A61K 31/495; A61K 31/18; A61K 31/165; A61K 31/4439; A61K 31/192; A61K 31/65; A61K 31/198; A61K 38/48; A61K 38/57; A61P 35/00

(52) U.S. Cl. CPC A61K 39/3955; A61K 31/496; A61K 31/495; A61K 31/18; A61K 31/165; A61P 35/00; A61K 31/192; A61K 31/65; A61K 31/198; A61K 38/4886; A61K 38/57; A61K 31/4439

(57) ABSTRACT

Materials and methods for inhibiting sPD-L1 production to prevent downregulation of the immune system and enhance the use of inhibitors of PD-1/PD-L1 interaction are provided herein. The materials and methods can be used in the treatment of cancer, for example.

### CROSS-REFERENCE TO RELATED APPLICATIONS

This application claims priority from U.S. Provisional Application Ser. No. 62/630,530, filed Feb. 14, 2018, and U.S. Provisional Application Ser. No. 62/664,748, filed Apr. 30, 2018. The disclosures of the prior applications are considered part of (and are incorporated by reference in) the disclosure of this application.

### STATEMENT AS TO FEDERALLY SPONSORED RESEARCH

This invention was made with government support under AI095239 and CA197878 awarded by the National Institutes of Health. The government has certain rights in the invention.

### TECHNICAL FIELD

This document relates to materials and methods for rescuing cancer patients from less than ideal response(s) to treatment with an inhibitor of PD-1/PD-L1 interaction (e.g., an anti-PD-1 antibody treatment or an anti-PD-L1 antibody treatment). For example, this document provides methods and materials that involve (a) administering a metallopeptidase inhibitor (e.g., an inhibitor of ADAM10 and/or ADAM17) to a mammal having cancer with limited, or no, responsiveness to treatment with an inhibitor of PD-1/PD-L1 interaction, in order to increase the effectiveness of an inhibitor of PD-1/PD-L1 interaction, and (b) administering an inhibitor of PD-1/PD-L1 interaction to the mammal to treat the cancer.

### BACKGROUND

Antibodies against programmed cell death protein 1 (PD-1) and programmed cell death protein ligand (PD-L1) are used for treating lung cancer, melanoma, and other cancers. In fact, anti-PD-1 monoclonal antibodies and anti-PD-L1 monoclonal antibodies have dramatically improved survival in melanoma, non-small cell lung cancer, Hodgkin's lymphoma, renal cell carcinoma, prostate cancer, bladder cancer, and colorectal cancer. Anti PD-1 antibodies such as pembrolizumab (MK-3475), nivolumab (BMS-936558), and pidilizumab can block ligands such as tumor-associated PD-L1 (also known as B7-H1) from interacting with PD-1 on tumor-reactive T cells, thus preventing tumor-induced T cell death. By blocking activation of this immune checkpoint, administered anti-PD-1 antibodies can improve a mammal's immune responses against tumors. Likewise, anti PD-L1 antibodies such as durvalumab, avelumab, and atezolizumab can block the interaction of PD-L1 with PD-1 and can improve a mammal's immune responses against tumors. The use of these inhibitors of PD-1/PD-L1 interaction is limited, however, because only 20-30% of patients respond to such treatments, and most tumors eventually develop resistance (Topalian et al., N Engl J Med, 366(26):2443-2454, 2012).

### SUMMARY

Mammals (e.g., humans) that do not respond to inhibitors of PD-1/PD-L1 interaction (e.g., anti-PD-1 antibodies or anti-PD-L1 antibodies) can express soluble PD-L1 (sPD-L1, also known as sB7-H1) in their serum (Dronca et al., J Clin Oncol, 35(15 Suppl):11534, 2017). The sPD-L1, which can be secreted by tumor cells (and some normal cells), can inhibit the effective use of anti-PD-1 antibodies by outcompeting the anti-PD-1 antibodies for binding to PD-1, and also can inhibit the effective use of anti-PD-L1 antibodies by providing an excess of a soluble antigen (i.e., sPD-L1) for the anti-PD-L1 antibodies such that little, or no, anti-PD-L1 antibody is available to bind to membrane bound PD-L1 on tumor cells. As described herein, metallopeptidases such as ADAM10 and ADAM17 (which are metallopeptidase enzymes that belong to the ADAM protein family of disintegrins and metalloproteases) in tumor cells are responsible for generating sPD-L1. Further, PD-L1 inhibitors (e.g., anti PD-L1 antibodies such as atezolizumab, or PD-1 analogues) can reduce sPD-L1 levels and rescue immune cell survival and activity.

Based at least in part on these findings, this document provides methods and materials for treating cancer patients with one or more metallopeptidase inhibitors (e.g., one or more ADAM10 inhibitors, one or more ADAM17 inhibitors, or a combination of both one or more ADAM10 inhibitors and one or more ADAM17 inhibitors) and one or more inhibitors of PD-1/PD-L1 interaction (e.g., one or more anti-PD-1 antibodies, one or more anti-PD-L1 antibodies, or a combination of both one or more anti-PD-1 antibodies and one or more anti-PD-L1 antibodies). This document also provides methods and materials for treating cancer patients with one or more PD-L1 inhibitors (e.g., one or more anti-PD-L1 antibodies such as avelumab, atezolizumab, or durvalumab, and/or one or more PD-1 receptors or receptor analogues such as rhPD1) and one or more inhibitors of PD-1/PD-L1 interaction (e.g., one or more anti-PD-1 antibodies, one or more anti-PD-L1 antibodies, or a combination of both one or more anti-PD-1 antibodies and one or more anti-PD-L1 antibodies). In some cases, reducing or preventing the ability of sPD-L1 to decrease the effectiveness of an inhibitor of PD-1/PD-L1 interaction to treat cancer as described herein can increase (e.g., quadruple) the number of patients who can receive benefit from an anti-PD-1 antibody and/or anti-PD-L1 antibody treatment protocol. Thus, the methods and materials described herein can provide improved responsiveness to immunotherapy, lengthened survival from cancer, and improved relief from symptoms. Such benefits can be experienced by cancer patients receiving one or more inhibitors of PD-1/PD-L1 interactions (e.g., an anti-PD-1 antibody and/or an anti-PD-L1 antibody), immunodeficient patients receiving one or more inhibitors of PD-1/PD-L1 interactions (e.g., an anti-PD-1 antibody and/or an anti-PD-L1 antibody), and other patients receiving or planning to receive one or more inhibitors of PD-1/PD-L1 interactions (e.g., an anti-PD-1 antibody and/or an anti-PD-L1 antibody).

In one aspect, this document features a method for enhancing effectiveness of an inhibitor of PD-1/PD-L1 interactions in a mammal identified as being in need thereof, where the method includes administering to the mammal (a) a metallopeptidase inhibitor and (b) an inhibitor of PD-1/PD-L1 interactions, wherein the metallopeptidase inhibitor is administered in an amount effective to reduce the level of sPD-L1 in the subject. The metallopeptidase inhibitor can be a small molecule (e.g., aderbasib, XL784, KP-457, GI254023X, Ro 32-3555, ARP 101, UK 370106, MMP-9 inhibitor-I (MMP9I), doxycycline, TAPI-0, TAPI-1, or TAPI-2). The metallopeptidase inhibitor can be an antibody [e.g., MEDI3622 or D1(A12)]. The metallopeptidase inhibitor can be an inhibitor of ADAM17 (e.g., MEDI3622, D1(A12), aderbasib, XL784, KP-457, ADAM17 prodomain, TIMP-3, TAPI-0, TAPI-1, or TAPI-2). The metallopeptidase inhibitor can be an inhibitor of ADAM10 (e.g., aderbasib, GI254023X, ADAM10 prodomain, TIMP-1, or XL784). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab). The mammal can be a human. The mammal can be identified as being PD-1 resistant or PD-L1 resistant (e.g., based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof). The mammal can be a cancer patient. The mammal can be identified as having a cancer selected from the group consisting of melanoma, non-small cell lung cancer (NSCLC), mesothelioma, esophageal cancer, gastric cancer, bladder cancer, squamous cell cancer, leukemia, lymphoma, renal cell carcinoma (RCC), prostate cancer, bladder cancer, and colorectal cancer.

In another aspect, this document features a method for immunomodulatory treatment, where the method includes administering to a mammal identified as being in need thereof (a) a metallopeptidase inhibitor and (b) an inhibitor of PD-1/PD-L1 interactions, wherein the metallopeptidase inhibitor is administered in an amount effective to reduce production of sPD-L1 in the subject, and wherein the inhibitor of PD-1/PD-L1 interactions is administered in an amount effective to modulate the activity of an immune cell within the mammal. The immune cell can be a CD8 T-cell, a CD4 T-cell, a dendritic cell, a natural killer cell, a macrophage, or a stromal cell. The metallopeptidase inhibitor can be a small molecule (e.g., aderbasib, XL784, KP-457, GI254023X, Ro 32-3555, ARP 101, UK 370106, MMP9I, doxycycline, TAPI-0, TAPI-1, or TAPI-2). The metallopeptidase inhibitor can be an antibody [e.g., MEDI3622 or D1(A12)]. The metallopeptidase inhibitor can be an inhibitor of ADAM17 (e.g., MEDI3622, D1(A12), aderbasib, XL784, KP-457, ADAM17 prodomain, TIMP-3, TAPI-0, TAPI-1, or TAPI-2). The metallopeptidase inhibitor can be an inhibitor of ADAM10 (e.g., aderbasib, GI254023X, ADAM10 prodomain, TIMP-1, or XL784). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab). The mammal can be a human. The mammal can be identified as being PD-1 resistant or PD-L1 resistant (e.g., based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof). The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

In another aspect, this document features a method for immunomodulatory treatment, where the method includes administering to a mammal identified as being in need thereof (a) an inhibitor of PD-L1 and (b) an inhibitor of PD-1/PD-L1 interactions, wherein the PD-L1 inhibitor is administered in an amount effective to reduce the amount of circulating sPD-L1 in the subject, and wherein the inhibitor of PD-1/PD-L1 interactions is administered in an amount effective to modulate the activity of an immune cell within the mammal. The immune cell can be a CD8 T-cell, a CD4 T-cell, a dendritic cell, a natural killer cell, a macrophage, or a stromal cell. The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The inhibitor of PD-1/PD-L1 interactions can be an anti-PD-1 antibody (e.g., pembrolizumab, nivolumab, or pidilizumab). The mammal can be a human. The mammal can be identified as being PD-1 resistant or PD-L1 resistant (e.g., based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof). The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

In another aspect, this document features a method for immunomodulatory treatment, where the method includes administering to a mammal identified as being in need thereof an inhibitor of PD-L1 rather than an inhibitor of PD-1, wherein the PD-L1 inhibitor is administered in an amount effective to reduce the amount of circulating sPD-L1 in the subject and to modulate the activity of an immune cell within the mammal. The immune cell can be a CD8 T-cell, a CD4 T-cell, a dendritic cell, a natural killer cell, a macrophage, or a stromal cell. The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The mammal can be a human. The mammal can be identified as being PD-1 resistant or PD-L1 resistant (e.g., based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof). The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

In another aspect, this document features a method for reducing the number of cancer cells within a mammal, where the method includes administering (a) a metallopeptidase inhibitor and (b) an inhibitor of PD-1/PD-L1 interactions to the mammal, and where the number of cancer cells within the mammal are reduced after the administering. The cancer cells can be melanoma cells, NSCLC cells, lymphoma cells, RCC cells, prostate cancer cells, bladder cancer cells, or colorectal cancer cells. The metallopeptidase inhibitor can be a small molecule (e.g., aderbasib, XL784, KP-457, GI254023X, Ro 32-3555, ARP 101, UK 370106, MMP9I, doxycycline, TAPI-0, TAPI-1, or TAPI-2). The metallopeptidase inhibitor can be an antibody [e.g., MEDI3622 or D1(A12)]. The metallopeptidase inhibitor can be an inhibitor of ADAM17 (e.g., MEDI3622, D1(A12), aderbasib, XL784, KP-457, ADAM17 prodomain, TIMP-3, TAPI-0, TAPI-1, or TAPI-2). The metallopeptidase inhibitor can be an inhibitor of ADAM10 (e.g., aderbasib, GI254023X, ADAM10 prodomain, TIMP-1, or XL784). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab). The method can include administering two or more metallopeptidase inhibitors to the mammal. The mammal can be a human. The mammal can be identified as being PD-1 resistant or PD-L1 resistant (e.g., based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof). The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

In another aspect, this document features a method for reducing the number of cancer cells within a mammal, where the method includes administering (a) a PD-L1 inhibitor and (b) an inhibitor of PD-1/PD-L1 interactions to the mammal, and where the number of cancer cells within the mammal are reduced after the administering. The cancer cells can be melanoma cells, NSCLC cells, lymphoma cells, RCC cells, prostate cancer cells, bladder cancer cells, or colorectal cancer cells. The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab). The method can include administering two or more PD-L1 inhibitors to the mammal. The mammal can be a human. The mammal can be identified as being PD-1 resistant or PD-L1 resistant (e.g., based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof). The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

In another aspect, this document features a method for reducing the number of cancer cells within a mammal, where the method includes administering an inhibitor of PD-L1 rather than an inhibitor of PD-1, and where the number of cancer cells within the mammal are reduced after the administering. The cancer cells can be melanoma cells, NSCLC cells, lymphoma cells, RCC cells, prostate cancer cells, bladder cancer cells, or colorectal cancer cells. The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The inhibitor of PD-1/PD-L1 interactions can be an anti-PD-L1 antibody (e.g., avelumab, atezolizumab, or durvalumab). The method can include administering two or more PD-L1 inhibitors to the mammal. The mammal can be a human. The mammal can be identified as being PD-1 resistant or PD-L1 resistant (e.g., based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof). The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

In still another aspect, this document features a method for treating a mammal identified as being resistant to an inhibitor of PD-1/PD-L1 interactions, where the method includes administering to the mammal (a) a metallopeptidase inhibitor and (b) an inhibitor of PD-1/PD-L1 interactions, wherein the metallopeptidase inhibitor is administered in an amount effective to reduce the resistance in the mammal. The metallopeptidase inhibitor can be a small molecule (e.g., aderbasib, XL784, KP-457, GI254023X, Ro 32-3555, ARP 101, UK 370106, MMP9I, doxycycline, TAPI-0, TAPI-1, or TAPI-2). The metallopeptidase inhibitor can be an antibody [e.g., MEDI3622 or D1(A12)]. The metallopeptidase inhibitor can be an inhibitor of ADAM17 (e.g., MEDI3622, D1(A12), aderbasib, XL784, KP-457, ADAM17 prodomain, TIMP-3, TAPI-0, TAPI-1, or TAPI-2). The metallopeptidase inhibitor can be an inhibitor of ADAM10 (e.g., aderbasib, GI254023X, ADAM10 prodomain, TIMP-1, or XL784). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab). The mammal can be a human. The mammal can be identified as being anti-PD-1 resistant. The mammal can be identified as being anti-PD-L1 resistant. The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

In another aspect, this document features a method for treating a mammal identified as being resistant to an inhibitor of PD-1/PD-L1 interactions, where the method includes administering to the mammal (a) a PD-L1 inhibitor and (b) an inhibitor of PD-1/PD-L1 interactions, wherein the PD-L1 inhibitor is administered in an amount effective to reduce the resistance in the mammal. The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab). The mammal can be a human. The mammal can be identified as being anti-PD-1 resistant. The mammal can be identified as being anti-PD-L1 resistant. The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

In yet another aspect, this document features a method for treating a mammal identified as being resistant to an inhibitor of PD-1/PD-L1 interactions, where the method includes administering to the mammal an inhibitor of PD-L1 rather than an inhibitor of PD-1, and where the PD-L1 inhibitor is administered in an amount effective to reduce the resistance in the mammal. The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The mammal can be a human. The mammal can be identified as being anti-PD-1 resistant. The mammal can be identified as being anti-PD-L1 resistant. The mammal can be a cancer patient. The mammal can be identified as having a cancer selected from the group consisting of melanoma, NSCLC, mesothelioma, esophageal cancer, gastric cancer, bladder cancer, squamous cell cancer, leukemia, lymphoma, RCC, prostate cancer, bladder cancer, and colorectal cancer.

This document also features a composition containing a metallopeptidase inhibitor and an inhibitor of PD-1/PD-L1 interactions. The composition can further contain a pharmaceutically acceptable carrier (e.g., water, saline solution, a binding agent, a filler, a lubricant, a disintegrate, or a wetting agent). The metallopeptidase inhibitor can be a small molecule (e.g., aderbasib, XL784, KP-457, GI254023X, Ro 32-3555, ARP 101, UK 370106, MMP9I, doxycycline, TAPI-0, TAPI-1, or TAPI-2). The metallopeptidase inhibitor can be an antibody [e.g., MEDI3622 or D1(A12)]. The metallopeptidase inhibitor can be an inhibitor of ADAM17 (e.g., MEDI3622, D1(A12), aderbasib, XL784, KP-457, ADAM17 prodomain, TIMP-3. TAPI-0, TAPI-1, or TAPI-2). The metallopeptidase inhibitor can be an inhibitor of ADAM10 (e.g., aderbasib, GI254023X, ADAM10 prodomain, TIMP-1, or XL784). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab).

This document also features a composition containing a PD-L1 inhibitor and an inhibitor of PD-1/PD-L1 interactions. The composition can further contain a pharmaceutically acceptable carrier (e.g., water, saline solution, a binding agent, a filler, a lubricant, a disintegrate, or a wetting agent). The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab).

In another aspect, this document features a kit containing a metallopeptidase inhibitor and an inhibitor of PD-1/PD-L1 interactions. The metallopeptidase inhibitor can be a small molecule (e.g., aderbasib, XL784, KP-457, GI254023X, Ro 32-3555, ARP 101, UK 370106, MMP9I, doxycycline, TAPI-0, TAPI-1, or TAPI-2). The metallopeptidase inhibitor can be an antibody [e.g., MEDI3622 or D1(A12)]. The metallopeptidase inhibitor can be an inhibitor of ADAM17 (e.g., MEDI3622, D1(A12), aderbasib, XL784, KP-457, ADAM17 prodomain, TIMP-3, TAPI-0, TAPI-1, or TAPI-2). The metallopeptidase inhibitor can be an inhibitor of ADAM10 (e.g., aderbasib, GI254023X, ADAM10 prodomain, TIMP-1, or XL784). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab).

In another aspect, this document features a kit containing a PD-L1 inhibitor and an inhibitor of PD-1/PD-L1 interactions. The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab).

In yet another aspect, this document features a system for immunomodulatory treatment, where the system includes a metallopeptidase inhibitor and an inhibitor of PD-1/PD-L1 interactions, wherein, when administered to a mammal with cancer, the metallopeptidase inhibitor and the inhibitor of PD-1/PD-L1 interactions are effective to increase immune system killing of cancer cells. The cancer cells can be melanoma cells, NSCLC cells, lymphoma cells, RCC cells, prostate cancer cells, bladder cancer cells, or colorectal cancer cells. The metallopeptidase inhibitor can be a small molecule (e.g., aderbasib, XL784, KP-457, GI254023X, Ro 32-3555, ARP 101, UK 370106, MMP9I, doxycycline, TAPI-0, TAPI-1, or TAPI-2). The metallopeptidase inhibitor can be an antibody [e.g., MEDI3622 or D1(A12)]. The metallopeptidase inhibitor can be an inhibitor of ADAM17 (e.g., MEDI3622, D1(A12), aderbasib, XL784, KP-457, ADAM17 prodomain, TIMP-3, TAPI-0, TAPI-1, or TAPI-2). The metallopeptidase inhibitor can be an inhibitor of ADAM10 (e.g., aderbasib, GI254023X, ADAM10 prodomain, TIMP-1, or XL784). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab).

In another aspect, this document features a system for immunomodulatory treatment, where the system includes a PD-L1 inhibitor and an inhibitor of PD-1/PD-L1 interactions, wherein, when administered to a mammal with cancer, the PD-L1 inhibitor and the inhibitor of PD-1/PD-L1 interactions are effective to increase immune system killing of cancer cells. The cancer cells can be melanoma cells, NSCLC cells, lymphoma cells, RCC cells, prostate cancer cells, bladder cancer cells, or colorectal cancer cells. The inhibitor of PD-L1 can be an anti-PD-L1 antibody (e.g., avelumab, atezolizumab, or durvalumab). The inhibitor of PD-L1 can be a PD-1 receptor analog (e.g., rhPD1). The inhibitor of PD-1/PD-L1 interactions can be an anti-PD-1 antibody (e.g., pembrolizumab, nivolumab, or pidilizumab). The inhibitor of PD-1/PD-L1 interactions can be an anti-PD-L1 antibody (e.g., avelumab, atezolizumab, or durvalumab).

In another aspect, this document features a system for immunomodulatory treatment, where the system includes one or more inhibitors of PD-L1 rather than one or more inhibitors of PD-1, wherein, when administered to a mammal with cancer, the one or more PD-L1 inhibitors are effective to increase immune system killing of cancer cells. The cancer cells can be melanoma cells, NSCLC cells, lymphoma cells, RCC cells, prostate cancer cells, bladder cancer cells, or colorectal cancer cells. The inhibitor of PD-L1 can be an anti-PD-L1 antibody (e.g., avelumab, atezolizumab, or durvalumab). The inhibitor of PD-L1 can be a PD-1 receptor analog (e.g., rhPD1).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram depicting modulation of the PD-1 checkpoint by an anti-PD-1 antibody or anti-PD-L1 antibody. As shown, a tumor cell expresses a ligand for PD-1 (PD-L1), and an effector immune cell expresses a PD-1 receptor. An anti-PD-1 antibody that engages PD-1 in an inhibitory capacity or an anti-PD-L1 antibody that engages PD-L1 in an inhibitory capacity can prevent downstream modulation of cell processes by the receiving immune effector cell.

FIG. 2 is a diagram depicting modulation of the PD-1 checkpoint through the action of a protease that cleaves PD-L1. As shown, a tumor cell expresses PD-L1, and also expresses a protease (e.g., a metallopeptidase such as a matrix metalloproteinase (MMP)) that cleaves the membrane-bound or intracellular PD-L1 to produce soluble PD-1 ligand (sPD-L1). An effect of this “decoy” is the modulation of the PD-1 immunomodulatory receptor on an immune cell in a way that can obviate or outcompete the effective use of anti-PD-1 antibodies (as shown) or saturate the effective use of anti-PD-L1 antibodies (not shown).

FIG. 3A is a diagram depicting modulation of the PD-1 checkpoint by protease inhibitors. A tumor cell that expresses PD-L1 is shown, as is a protease that normally would cleave PD-L1 to generate sPD-L1. An added protease inhibitor, however, prevents that protease from cleaving PD-L1, thus reducing or preventing sPD-L1 production. This has the downstream effect of preventing sPD-L1 engagement with the immunomodulatory PD-1 receptor, thus facilitating the effectiveness of an anti-PD-1 antibody (as shown). FIG. 3B is a diagram depicting modulation of the PD-1 checkpoint by protease inhibitors. A tumor cell that expresses PD-L1 is shown, as is a protease that normally would cleave PD-L1 to generate sPD-L1. An added protease inhibitor, however, prevents the protease from cleaving PD-L1, thus reducing or preventing sPD-L1 production. This has the downstream effect of preventing an abundance of sPD-L1 that would otherwise saturate the effective use of anti-PD-L1 antibodies such that few, if any, anti-PD-L1 antibodies would be available to bind to PD-L1.

FIGS. 4A-4G are a series of graphs plotting levels of sPD-L1 production by tumor cells treated with various protease inhibitors, demonstrating that ADAM17 and related MMPs are principally responsible for production of sPD-L1. Tumor cells were treated with PBS (placebo) or with various protease inhibitors, including MMP inhibitors TAPI-2 and TAPI-0, aspartic protease inhibitor Pepstatin, cysteine protease inhibitor E-64, serine protease inhibitor Aprotinin, combinations of these inhibitors, and an array of other MMP inhibitors. Cell supernatants were queried for sPD-L1 by ELISA. FIG. 4A, Karpas-299 (lymphoma) cells were treated with the indicated protease inhibitors. FIG. 4B, A786-0 (renal cell carcinoma) cells were treated with the indicated protease inhibitors. FIG. 4C, Dul45 cells were treated with the indicated protease inhibitors. FIG. 4D, Mel-B7H1 (transgenic melanoma) cells were treated with the indicated protease or metalloprotease inhibitors. FIG. 4E is a graph plotting sPD-L1 production induced by interferon treatment or high cell titer in A786-0 cells. Cells grown at 1 MM/ml in control DMSO produced little sPD-L1. Interferon treatment increased sPD-L1 production significantly (mean 263 pg/ml vs. 923 pg/ml, p=7.18⁻⁵). Additional treatment with TAPI-0 reduced sPD-L1 production significantly (mean 923 pg/ml vs. 0 pg/ml, p=2.65⁻⁵). A786-0 cells grown at 10 MM/ml in control DMSO produced sPD-L1, which was reduced significantly by ADAM10/ADAM17 inhibitor aderbasib (mean 886 pg/ml vs 276 pg/ml, p=0.00018). n=5. FIG. 4F is a graph plotting the amount of sPD-L1 in supernatants of Karpas-299 cells treated with control or with varying concentrations of the ADAM10/17 inhibitor aderbasib. FIG. 4G is a graph plotting the amount of sPD-L1 in supernatants of A459 cells—a NSCLC line that does not secrete appreciable amounts of sPD-L1—treated with control or with exogenous ADAM17.

FIGS. 5A-5D show that sPDL1 induces human CD8+ T cell death in vitro, and sPD-L1 and pembrolizumab compete for effect on CD8+ T cells. FIG. 5A is a graph plotting survival of CD8+ human T cells cocultured in the presence of PBS control or 5 μg/ml or 10 μg/ml recombinant sPD-L1. Cell survival (assessed by trypan blue staining) was reduced in cultures containing 5 μg/ml recombinant PD-L1 (p<0.0001) and 10 μg/ml recombinant PD-L1 (p<0.001); n=3. FIG. 5B is a graph plotting survival of CD8+ human T cells cultured in the presence of PBS control or sPD-L1-rich Karpas-299 cell line supernatant, in the presence or absence of PD1 or PD-L1 checkpoint inhibitors. Cell survival was measured by trypan blue staining. Karpas-299 supernatant significantly decreased CD8+ T cell survival versus PBS control (p=0.005). Therapeutic doses of PD1 checkpoint inhibitors nivolumab (p=0.011), atezolizumab (p=0.022), and pembrolizumab (p=0.001) did not rescue CD8+ T cells; n=3. FIGS. 5C and 5D are a series of flow cytometry plots indicating survival of CD8+ T cells treated with various cell extracts and an anti-PD-1 antibody (nivolumab or pembrolizumab) or an anti-PD-L1 antibody (atezolizumab). FIG. 5C, CD8+ T cells isolated from healthy subjects were incubated with sPD-L1-rich supernatants from Karpas-299 cells, supernatants from Karpas-299 cells treated with inhibitor TAPI-0, supernatants from Karpas-299 cells “spiked” with TAPI-0 after isolation, and controls (top), and TMRE uptake/Annexin-V binding was measured by flow cytometry to assess the level of CD8+ T cell survival. CD8+ T cells also were incubated with supernatants from Karpas-299 cells, plus the indicated doses of anti-PD-1 and anti-PD-L1 antibodies (bottom). FIG. 5D, CD8+ T cells were incubated with PD-L1-rich supernatants from A787-0 RCC cells and the indicated concentrations of pembrolizumab.

FIGS. 6A-6H show that primary melanoma ADAM10 and ADAM17 expression correlates negatively with PD-L1 expression. Each figure contains images showing primary melanoma samples stained for ADAM10, ADAM17, and PD-L1, as indicated, by immunohistochemistry (IHC). Positive-staining samples as determined by a pathologist are marked in the upper-right corner with an asterisk (*), while other samples are negative. Fisher's exact test showed a significant negative correlation between ADAM10 or ADAM17 positivity and PD-L1 positivity (p=0.01786).

FIGS. 7A and 7B are flow cytometry plots and graphs showing that cell PD-L1 expression is restored by treatment with an inhibitor of ADAM10 and/or ADAM17, and is decreased by treatment with exogenous recombinant ADAM10 or ADAM17. Cells were incubated in the presence of the indicated treatments, and PD-L1 flow cytometry was performed. FIGS. 7A and 7B show the results of flow cytometry using Karpas-299 cells, which are known to shed soluble PD-L1, while FIGS. 7C and 7D show results using A549 cells, which do not shed appreciable amounts of soluble PD-L1.

Throughout the figures: *** P<0.001, ** P<0.01, * P<0.05, #P<0.10.

### DETAILED DESCRIPTION

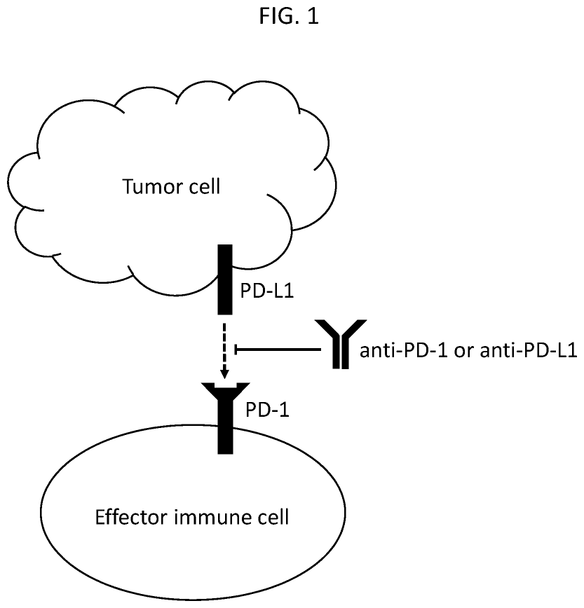

PD-1 (also referred to as PD-1 receptor, CD279, and SLEB2) is an immunomodulatory receptor that can engage PD-L1 (also referred to as PD-1 ligand, CD274, B7-H1) and PD-L2 (also referred to as PDCD1LG2 and B7-DC). The PD-1 receptor is a checkpoint protein that is expressed on the surface of T cells. PD-1 typically acts to prevent T cells from attacking other cells in the body; such prevention can be triggered when PD-1 is bound by its ligand, PD-L1 (also referred to as B7-H1). PD-L1 is expressed by some normal cells and also by cancer cells. As depicted in FIG. 1, the PD-1 checkpoint can be modulated by anti-PD-1 antibodies or anti-PD-L1 antibodies, which can engage PD-1 or PD-L1, respectively, and prevent interaction between the two molecules, thus reducing or preventing downstream modulation of cell processes by the receiving immune effector cell (e.g., a CD8 T-cell, a CD4 T-cell, a dendritic cell, a natural killer cell, a macrophage, or a stromal cell).

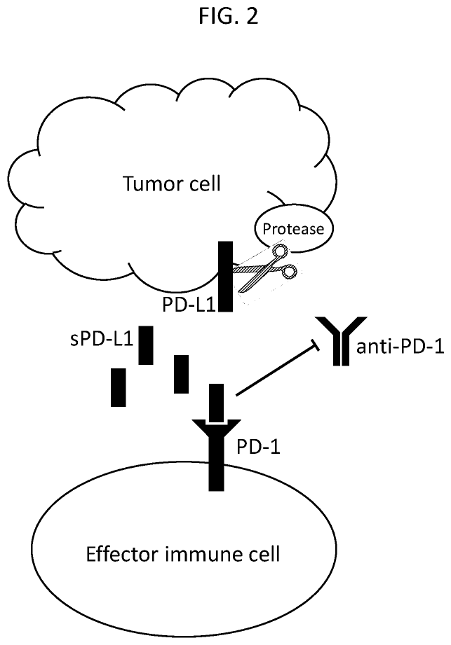

In tumor cells and some normal cells, PD-L1 is cleaved to release soluble PD-L1 into the serum. sPD-L1 can compete with uncleaved PD-L1, and also with anti-PD-1 antibodies, for binding to PD-1. FIG. 2 illustrates such modulation of the PD-1 checkpoint. As shown, a tumor cell expresses PD-L1 and also expresses a protease that cleaves the membrane-bound or intracellular PD-L1 to generate sPD-L1. Interaction of the sPD-L1 with PD-1 on an immune cell can interfere with the inhibitory action of an anti-PD-1 antibody. Likewise, accumulation of sPD-L1 can saturate the effectiveness of anti-PD-L1 antibodies such that few, if any, anti-PD-L1 antibodies are available to bind to PD-L1 present on cancer cells and block the PD-1/PD-L1 interaction. This, in turn in both cases, prevents the immune cell from attacking cancer cells.

As used herein, the terms “PD-1 resistant,” “anti-PD-1 antibody resistant,” and “anti-PD-1 non-responder” are used interchangeably to refer to cells, tumors, and/or subjects that do not respond, or that have a reduced response, to treatments targeted to PD-1 (e.g., anti-PD-1 antibodies) due to, for example, interference from sPD-L1.

As used herein, the terms “PD-L1 resistant,” “anti-PD-L1 antibody resistant,” and “anti-PD-L1 non-responder” are used interchangeably to refer to cells, tumors, and/or subjects that do not respond, or that have a reduced response, to treatments targeted to PD-L1 (e.g., anti-PD-L1 antibodies) due to, for example, interference from sPD-L1.

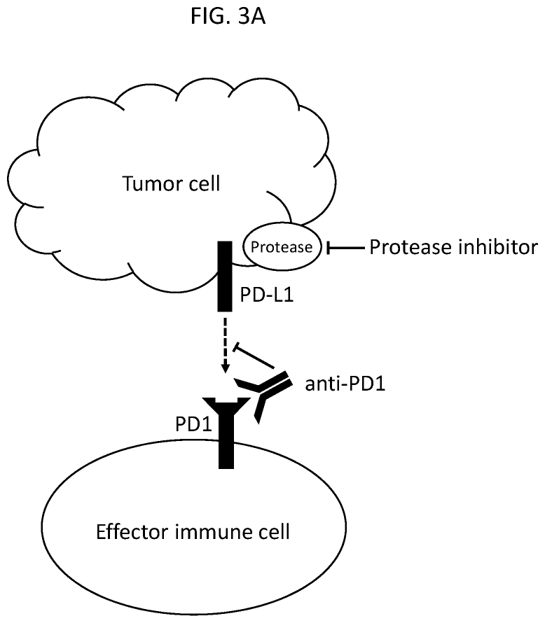

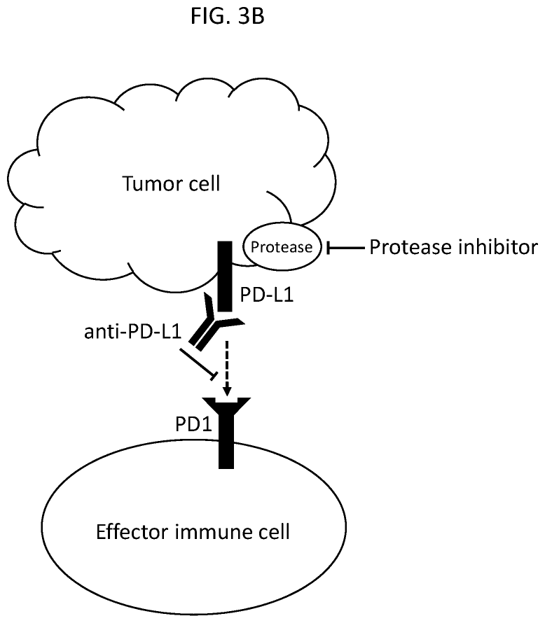

As described herein, protease inhibitors (e.g., metallopeptidase inhibitors such as MMP inhibitors, ADAM10 inhibitors, and/or ADAM17 inhibitors) can be used to inhibit or prevent cleavage of PD-L1 to form sPD-L1, thus reducing or removing the interference of sPD-L1 with the effectiveness of anti-PD-1 antibodies and/or the effectiveness of anti-PD-L1 antibodies, thereby increasing their effectiveness. FIGS. 3A and 3B depict therapeutic methods that can be used to modulate the PD-1 checkpoint by using such protease inhibitors. The protease inhibitor can prevent protease activity, thus reducing or preventing sPD-L1 production, which can have the downstream effects of (a) reducing or preventing sPD-L1 engagement with PD-1 and enhancing the effectiveness of an anti-PD-1 antibody, and (b) reducing or preventing sPD-L1 from binding to administered anti-PD-L1 antibodies and enhancing the effectiveness of an anti-PD-L1 antibody.

Any appropriate metallopeptidase inhibitor can be used as described herein to reduce or prevent the generation of sPD-L1 from PD-L1 within a mammal (e.g., a human). For example, MMP inhibitors can be used as described herein to reduce or prevent the generation of sPD-L1 from PD-L1 within a mammal (e.g., a human). In some cases, ADAM10 inhibitors, ADAM17 inhibitors, or combinations thereof can be used as described herein to reduce or prevent the generation of sPD-L1 from PD-L1 within a mammal (e.g., a human). In some cases, a metallopeptidase inhibitor, MMP inhibitor, ADAM10 inhibitor, or ADAM17 inhibitor described herein can be a small molecule that inhibits enzymatic activity of the protease, an antibody that binds to the protease and inhibits enzymatic activity of the protease, or a nucleic acid molecule (e.g., a siRNA molecule, an antisense molecule, and/or an RNA interference molecule) that reduces or prevents expression of the protease. Examples of metallopeptidase inhibitors that can be used as described herein to reduce the level of sPD-L1 production within a mammal (e.g., a human) include, without limitation, MEDI3622 (an anti-ADAM17 monoclonal antibody), D1(A12) (a humanized anti-ADAM17 monoclonal antibody), and the small molecules TAPI-0, TAPI-1, TAPI-2, aderbasib, XL784, KP-457, GI254023X, Ro 32-3555, ARP 101, UK 370106, MMP9I, and doxycycline. Examples of MMP inhibitors that can be used as described herein to reduce the level of sPD-L1 production within a mammal (e.g., a human) include, without limitation, TAPI-2, TAPI-0, GI254023X, Ro 32-3555, ARP 101, UK 370106, MMP9I, and doxycycline. Examples of ADAM10 inhibitors that can be used as described herein to reduce the level of sPD-L1 production within a mammal (e.g., a human) include, without limitation, aderbasib, GI254023X, ADAM10 prodomain, TIMP-1, and XL784. Examples of inhibitors of ADAM17 (also referred to as tumor necrosis factor converting enzyme or TACE) that can be used as described herein to reduce the level of sPD-L1 production within a mammal (e.g. a human) include, without limitation, MEDI3622, D1(A12), aderbasib, XL784, KP-457, ADAM17 prodomain, TIMP-3, TAPI-0, TAPI-1, and TAPI-2. Examples of some of the above inhibitors and their commercial sources are listed in TABLE 1.

TABLE 1
Metallopeptidase (ADAM10/ADAM17) inhibitors

| Name | Type | Source |
|---|---|---|
| INCB7839 (Aderbasib) | Small molecule | Incyte Corp. |
| XL784 | Small molecule | Exelixis, Inc. |
| MEDI3622 | Monoclonal antibody | MedImmune/AstraZeneca |
| D1(A12) | Humanized monoclonal antibody | Cambridge |
| KP-457 | Small molecule | Kaken Pharmaceutical |
| TAPI-0 | Small molecule | Teva |
| TAPI-1 | Small molecule | Teva |
| TAPI-2 | Small molecule | Teva |
| TIMP-1 | Polypeptide | Sino Biological |
| TIMP-3 | Polypeptide | Sino Biological |

In some cases, an inhibitor that can be used as described herein to reduce the level of sPD-L1 production within a mammal (e.g., a human) can be an anti-ADAM10 antibody, an anti-ADAM17 antibody, a combination of ADAM10 and ADAM17 antibodies, or a bispecific antibody that can bind to both ADAM10 and ADAM17. Any appropriate method can be used to generate antibodies against ADAM10 and ADAM17.

Representative human ADAM10 amino acid and nucleotide sequences are set forth in SEQ ID NOS:1 and 2, respectively:

(SEQ ID NO: 1)
MVLLRVLILLLSWAAGMGGQYGNPLNKYIRHYEGLSYNVDSLHQKHQRAK
RAVSHEDQFLRLDFHAHGRHFNLRMKRDTSLFSDEFKVETSNKVLDYDTS
HIYTGHIYGEEGSFSHGSVIDGRFEGFIQTRGGTFYVEPAERYIKDRTLP
FHSVIYHEDDINYPHKYGPQGGCADHSVFERMRKYQMTGVEEVTQIPQEE
HAANGPELLRKKRTTSAEKNTCQLYIQTDHLFFKYYGTREAVIAQISSHV
KAIDTIYQTTDFSGIRNISFMVKRIRINTTADEKDPTNPFRFPNIGVEKF
LELNSEQNHDDYCLAYVFTDRDFDDGVLGLAWVGAPSGSSGGICEKSKLY
SDGKKKSLNTGIITVQNYGSHVPPKVSHITFAHEVGHNFGSPHDSGTECT
PGESKNLGQKENGNYIMYARATSGDKLNNNKFSLCSIRNISQVLEKKRNN
CFVESGQPICGNGMVEQGEECDCGYSDQCKDECCFDANQPEGRKCKLKPG
KQCSPSQGPCCTAQCAFKSKSEKCRDDSDCAREGICNGFTALCPASDPKP
NFTDCNRHTQVCINGQCAGSICEKYGLEECTCASSDGKDDKELCHVCCMK
KMDPSTCASTGSVQWSRHFSGRTITLQPGSPCNDFRGYCDVFMRCRLVDA
DGPLARLKKAIFSPELYENIAEWIVAHWWAVLLMGIALIMLMAGFIKICS
VHTPSSNPKLPPPKPLPGTLKRRRPPQPIQQPQRQRPRESYQMGHMRR
(SEQ ID NO: 2)
atggtgttgctgagagtgttaattctgctcctctcctgggcggcggggat
gggaggtcagtatgggaatcctttaaataaatatatcagacattatgaag
gattatcttacaatgtggattcattacaccaaaaacaccagcgtgccaaa
agagcagtctcacatgaagaccaatttttacgtctagatttccatgccca
tggaagacatttcaacctacgaatgaagagggacacttcccttttcagtg
atgaatttaaagtagaaacatcaaataaagtacttgattatgatacctct
catatttacactggacatatttatggtgaagaaggaagttttagccatgg
gtctgttattgatggaagatttgaaggattcatccagactcgtggtggca
cattttatgttgagccagcagagagatatattaaagaccgaactctgcca
tttcactctgtcatttatcatgaagatgatattaactatccccataaata
cggtcctcaggggggctgtgcagatcattcagtatttgaaagaatgagga
aataccagatgactggtgtagaggaagtaacacagatacctcaagaagaa
catgctgctaatggtccagaacttctgaggaaaaaacgtacaacttcagc
tgaaaaaaatacttgtcagctttatattcagactgatcatttgttcttta
aatattacggaacacgagaagctgtgattgcccagatatccagtcatgtt
aaagcgattgatacaatttaccagaccacagacttctccggaatccgtaa
catcagtttcatggtgaaacgcataagaatcaatacaactgctgatgaga
aggaccctacaaatcctttccgtttcccaaatattggtgtggagaagttt
ctggaattgaattctgagcagaatcatgatgactactgtttggcctatgt
cttcacagaccgagattttgatgatggcgtacttggtctggcttgggttg
gagcaccttcaggaagctctggaggaatatgtgaaaaaagtaaactctat
tcagatggtaagaagaagtccttaaacactggaattattactgttcagaa
ctatgggtctcatgtacctcccaaagtctctcacattactttgctcacga
agttggacataactttggatccccacatgattctggaacagagtgcacac
caggagaatctaagaatttgggtcaaaaagaaaatggcaattacatcatg
tatgcaagagcaacatctggggacaaacttaacaacaataaattctcact
ctgtagtattagaaatataagccaagttcttgagaagaagagaaacaact
gttttgttgaatctggccaacctatttgtggaaatggaatggtagaacaa
ggtgaagaatgtgattgtggctatagtgaccagtgtaaagatgaatgctg
cttcgatgcaaatcaaccagagggaagaaaatgcaaactgaaacctggga
aacagtgcagtccaagtcaaggtccttgttgtacagcacagtgtgcattc
aagtcaaagtctgagaagtgtcgggatgattcagactgtgcaagggaagg
aatatgtaatggcttcacagctctctgcccagcatctgaccctaaaccaa
acttcacagactgtaataggcatacacaagtgtgcattaatgggcaatgt
gcaggttctatctgtgagaaatatggcttagaggagtgtacgtgtgccag
ttctgatggcaaagatgataaagaattatgccatgtatgctgtatgaaga
aaatggacccatcaacttgtgccagtacagggtctgtgcagtggagtagg
cacttcagtggtcgaaccatcaccctgcaacctggatccccttgcaacga
ttttagaggttactgtgatgttttcatgcggtgcagattagtagatgctg
atggtcctctagctaggcttaaaaaagcaatttttagtccagagctctat
gaaaacattgctgaatggattgtggctcattggtgggcagtattacttat
gggaattgctctgatcatgctaatggctggatttattaagatatgcagtg
ttcatactccaagtagtaatccaaagttgcctcctcctaaaccacttcca
ggcactttaaagaggaggagacctccacagcccattcagcaaccccagcg
tcagcggccccgagagagttatcaaatgggacacatgagacgctaa

A representative ADAM10 prodomain sequence is set forth in amino acids 20-213 of SEQ ID NO:1 (underlined).

Examples of representative human ADAM17 amino acid and nucleotide sequences are set forth in SEQ ID NOS:3 and 4, respectively:

(SEQ ID NO: 3)
MRQSLLFLTSVVPFVLAPRPPDDPGFGPHQRLEKLDSLLSDYDILSLSNI
QQHSVRKRDLQTSTHVETLLTFSALKRHFKLYLTSSTERFSQNFKVVVVD
GKNESEYTVKWQDFFTGHVVGEPDSRVLAHIRDDDVIIRINTDGAEYNIE
PLWRFVNDTKDKRMLVYKSEDIKNVSRLQSPKVCGYLKVDNEELLPKGLV
DREPPEELVHRVKRRADPDPMKNTCKLLVVADHRFYRYMGRGEESTTTNY
LIHTDRAN
(SEQ ID NO: 4)
atgaggcagtctctcctattcctgaccagcgtggttcctttcgtgctggc
gccgcgacctccggatgacccgggcttcggcccccaccagagactcgaga
agcttgattctttgctctcagactacgatattctctctttatctaatatc
cagcagcattcggtaagaaaaagagatctacagacttcaacacatgtaga
aacactactaactttttcagctttgaaaaggcattttaaattatacctga
catcaagtactgaacgtttttcacaaaatttcaaggtcgtggtggtggat
ggtaaaaacgaaagcgagtacactgtaaaatggcaggacttcttcactgg
acacgtggttggtgagcctgactctagggttctagcccacataagagatg
atgatgttataatcagaatcaacacagatggggccgaatataacatagag
ccactttggagatttgttaatgataccaaagacaaaagaatgttagttta
taaatctgaagatatcaagaatgtttcacgtttgcagtctccaaaagtgt
gtggttatttaaaagtggataatgaagagttgctcccaaaagggttagta
gacagagaaccacctgaagagcttgttcatcgagtgaaaagaagagctga
cccagatcccatgaagaacacgtgtaaattattggtggtagcagatcatc
gcttctacagatacatgggcagaggggaagagagtacaactacaaattac
ttaatacacagatagagctaattga

Additional examples of human ADAM17 amino acid and nucleotide sequences are set forth in SEQ ID NOS:5 and 6, respectively:

(SEQ ID NO: 5)
MRQSLLFLTSVVPFVLAPRPPDDPGEGPHQRLEKLDSLLSDYDILSLSNI
QQHSVRKRDLQTSTHVETLLTFSALKRHFKLYLTSSTERFSQNFKVVVVD
GKNESEYTVKWQDFFTGHVVGEPDSRVLAHIRDDDVIIRINTDGAEYNIE
PLWRFVNDTKDKRMLVYKSEDIKNVSRLQSPKVCGYLKVDNEELLPKGLV
DREPPEELVHRVKRRADPDPMKNTCKLLVVADHRFYRYMGRGEESTTTNY
LIELIDRVDDIYRNTSWDNAGFKGYGIQIEQIRILKSPQEVKPGEKHYNM
AKSYPNEEKDAWDVKMLLEQFSFDIAEEASKVCLAHLFTYQDFDMGTLGL
AYVGSPRANSHGGVCPKAYYSPVGKKNIYLNSGLTSTKNYGKTILTKEAD
LVTTHELGHNFGAEHDPDGLAECAPNEDQGGKYVMYPIAVSGDHENNKMF
SNCSKQSIYKTIESKAQECFQERSNKVCGNSRVDEGEECDPGIMYLNNDT
CCNSDCTLKEGVQCSDRNSPCCKNCQFETAQKKCQEAINATCKGVSYCTG
NSSECPPPGNAEDDTVCLDLGKCKDGKCIPFCEREQQLESCACNETDNSC
KVCCRDLSGRCVPYVDAEQKNLFLRKGKPCTVGFCDMNGKCEKRVQDVIE
RFWDFIDQLSINTEGKFLADNIVGSVLVFSLIFWIPFSILVHCVDKKLDK
QYESLSLFHPSNVEMLSSMDSASVRIIKPFPAPQTPGRLQPAPVIPSAPA
APKLDHQRMDTIQEDPSTDSHMDEDGFEKDPFPNSSTAAKSFEDLTDHPV
TRSEKAASFKLQRQNRVDSKETEC
(SEQ ID NO: 6)
atgaggcagtactcctattcctgaccagcgtggttcctttcgtgctggcg
ccgcgacctccggatgacccgggcttcggcccccaccagagactcgagaa
gcttgattattgactcagactacgatattactattatctaatatccagca
gcattcggtaagaaaaagagatctacagacttcaacacatgtagaaacac
tactaactttttcagctttgaaaaggcattttaaattatacctgacatca
agtactgaacgtttttcacaaaatttcaaggtcgtggtggtggatggtaa
aaacgaaagcgagtacactgtaaaatggcaggacttcttcactggacacg
tggttggtgagcctgactctagggttctagcccacataagagatgatgat
gttataatcagaatcaacacagatggggccgaatataacatagagccact
ttggagatttgttaatgataccaaagacaaaagaatgttagtttataaat
ctgaagatatcaagaatgtttcacgtttgcagtctccaaaagtgtgtggt
tatttaaaagtggataatgaagagttgctcccaaaagggttagtagacag
agaaccacctgaagagcttgttcatcgagtgaaaagaagagctgacccag
atcccatgaagaacacgtgtaaattattggtggtagcagatcatcgcttc
tacagatacatgggcagaggggaagagagtacaactacaaattacttaat
agagctaattgacagagttgatgacatctatcggaacacttcatgggata
atgcaggttttaaaggctatggaatacagatagagcagattcgcattctc
aagtctccacaagaggtaaaacctggtgaaaagcactacaacatggcaaa
aagttacccaaatgaagaaaaggatgcttgggatgtgaagatgttgctag
agcaatttagattgatatagctgaggaagcatctaaagtttgcttggcac
accttttcacataccaagattttgatatgggaactcttggattagcttat
gttggctctcccagagcaaacagccatggaggtgtttgtccaaaggctta
ttatagcccagttgggaagaaaaatatctatttgaatagtggtttgacga
gcacaaagaattatggtaaaaccatccttacaaaggaagctgacctggtt
acaactcatgaattgggacataattttggagcagaacatgatccggatgg
tctagcagaatgtgccccgaatgaggaccagggagggaaatatgtcatgt
atcccatagctgtgagtggcgatcacgagaacaataagatgttttcaaac
tgcagtaaacaatcaatctataagaccattgaaagtaaggcccaggagtg
ttttcaagaacgcagcaataaagtttgtgggaactcgagggtggatgaag
gagaagagtgtgatcctggcatcatgtatctgaacaacgacacctgctgc
aacagcgactgcacgttgaaggaaggtgtccagtgcagtgacaggaacag
tccttgctgtaaaaactgtcagtttgagactgcccagaagaagtgccagg
aggcgattaatgctacttgcaaaggcgtgtcctactgcacaggtaatagc
agtgagtgcccgcctccaggaaatgctgaagatgacactgtttgcttgga
tcttggcaagtgtaaggatgggaaatgcatccdttctgcgagagggaaca
gcagctggagtcctgtgcatgtaatgaaactgacaactcctgcaaggtgt
gctgcagggacctttctggccgctgtgtgccctatgtcgatgctgaacaa
aagaacttatttttgaggaaaggaaagccctgtacagtaggattttgtga
catgaatggcaaatgtgagaaacgagtacaggatgtaattgaacgatttt
gggatttcattgaccagctgagcatcaatacttttggaaagtttttagca
gacaacatcgttgggtctgtcctggttttctccttgatattttggattcc
tttcagcattcttgtccattgtgtggataagaaattggataaacagtatg
aatctctgtctctgtttcaccccagtaacgtcgaaatgctgagcagcatg
gattctgcatcggttcgcattatcaaaccdttcctgcgccccagactcca
ggccgcctgcagcctgcccctgtgatcccttcggcgccagcagctccaaa
actggaccaccagagaatggacaccatccaggaagaccccagcacagact
cacatatggacgaggatgggtttgagaaggaccccttcccaaatagcagc
acagctgccaagtcatttgaggatctcacggaccatccggtcaccagaag
tgaaaaggctgcctcctttaaactgcagcgtcagaatcgtgttgacagca
aagaaacagagtgctaa

Representative ADAM17 prodomain sequences are set forth in amino acids 18-214 of SEQ ID NOS:3 and 5 (underlined).

As used herein, the term “antibody” refers to any immunoglobulin or antibody (e.g., human, hamster, feline, mouse, cartilaginous fish, or camelid antibodies), and any derivative or conjugate thereof, that specifically binds to an antigen. Non-limiting examples of antibodies include monoclonal antibodies, polyclonal antibodies, humanized antibodies, multi-specific antibodies (e.g., bispecific antibodies), single-chain antibodies (e.g., single-domain antibodies, camelid antibodies, and cartilaginous fish antibodies), chimeric antibodies, feline antibodies, and felinized antibodies. The term “antibody” also includes antibody derivatives and conjugates (e.g., an antibody conjugated to a stabilizing protein, a detectable moiety, or a therapeutic agent).

Antigen binding fragments of antibodies also can be used in the methods provided herein. An “antigen binding fragment” is any portion of a full-length antibody that contains at least one variable domain (e.g., a variable domain of a mammalian (e.g., feline, human, hamster, or mouse) heavy or light chain immunoglobulin, a camelid variable antigen binding domain (VHH), or a cartilaginous fish immunoglobulin new antigen receptor (Ig-NAR) domain) that is capable of specifically binding to an antigen. Non-limiting examples of antibody fragments include Fab, Fab′, F(ab′)2, and Fv fragments, diabodies, linear antibodies, and multi-specific antibodies formed from antibody fragments.

An Fv fragment is the minimum antibody fragment that contains a complete antigen recognition and binding site. This region consists of a dimer of one heavy chain variable domain and one light chain variable domain in tight, non-covalent association. It is in this configuration that the three complementary determining regions (CDRs) of each variable domain interact to define an antigen binding site on the surface of the VH-VL dimer. A “complementary determining region” or “CDR” is a region within an immunoglobulin (a heavy or light chain immunoglobulin) that forms part of an antigen binding site in an antibody or antigen binding fragment thereof. Heavy chain and light chain immunoglobulins each contain three CDRs, referred to as CDR1, CDR2, and CDR3. In any antibody or antigen binding fragment, the three CDRs from the heavy chain immunoglobulin and the three CDRs from the light chain immunoglobulin together form an antigen binding site in the antibody or antigen binding fragment thereof. The Kabat Database is one system used in the art to number CDR sequences present in a light chain immunoglobulin or a heavy chain immunoglobulin.

Collectively, the six CDR's confer antigen binding specificity to the antibody. However, even a single variable domain (or half of an Fv comprising only three CDR's specific for an antigen) has the ability to recognize and bind the antigen, although usually at a lower affinity than the entire binding site. The “Fab fragment” also contains the constant domain of the light chain and the first constant domain (CH1) of the heavy chain. The “Fab fragment” differs from the “Fab′ fragment” by the addition of a few residues at the carboxy terminus of the heavy chain CH1 domain, including one or more cysteines from the antibody hinge region. The “F(ab′)2 fragment” originally is produced as a pair of “Fab′ fragments” which have hinge cysteines between them. Methods of preparing such antibody fragments include, without limitation, papain or pepsin digestion.

An antibody can be of the IgA-, IgD-, IgE, IgG- or IgM-type, including IgG- or IgM-types such as, without limitation, IgG1-, IgG2-, IgG3-, IgG4-, IgM1- and IgM2-types. For example, in some cases, the antibody is of the IgG1-, IgG2- or IgG4-type. In some embodiments, antibodies can be fully human or humanized antibodies.

By “human antibody” is meant an antibody that is encoded by a nucleic acid (e.g., a rearranged human immunoglobulin heavy or light chain locus) present in the genome of a human. In some embodiments, a human antibody can be produced in a human cell culture (e.g., feline hybridoma cells). In some embodiments, a human antibody can be produced in a non-human cell (e.g., a mouse or hamster cell line). In some cases, a human antibody can be produced in a bacterial or yeast cell.

The term “humanized antibody” refers to a human antibody that contains minimal sequence derived from non-human (e.g., mouse, hamster, rat, rabbit, or goat) immunoglobulin. Humanized antibodies generally are chimeric or mutant monoclonal antibodies from mouse, rat, hamster, rabbit or other species, bearing human constant and/or variable region domains or specific changes. In non-limiting examples, humanized antibodies are human antibodies (recipient antibody) in which hypervariable region (HVR) residues of the recipient antibody are replaced by HVR residues from a non-human species (donor) antibody, such as a mouse, rat, rabbit, or goat antibody having the desired specificity, affinity, and capacity. In some embodiments, Fv framework residues of the human immunoglobulin can be replaced by corresponding non-human residues. In some embodiments, humanized antibodies can contain residues that are not found in the recipient antibody or in the donor antibody. Such modifications can be made to refine antibody performance, for example.

In some embodiments, a humanized antibody can contain substantially all of at least one, and typically two, variable domains, in which all or substantially all of the hypervariable loops (CDRs) correspond to those of a non-human immunoglobulin, while all or substantially all of the framework regions are those of a human immunoglobulin sequence. A humanized antibody also can contain at least a portion of an immunoglobulin constant (Fc) region, typically that of a human immunoglobulin.

As used herein, the term “single-domain antibody” refers to a polypeptide that contains one camelid VHH or at least one cartilaginous fish Ig-NAR domain that is capable of specifically binding to an antigen. Non-limiting examples of single-domain antibodies are described, for example, in U.S. Publication No. 2010/0092470.

An antibody or antigen binding fragment thereof “specifically binds” to a particular antigen, e.g., ADAM10 or ADAM17, when it binds to that antigen in a sample, and does not recognize and bind, or recognizes and binds to a lesser extent, other molecules in the sample. In some cases, an antibody or an antigen binding fragment can selectively bind to an epitope with an affinity (KD) of about, for example, 1×10⁻⁶ M or less (e.g., about 1×10⁻⁷ M, about 1×10⁻⁸M, about 1×10⁻⁹ M, or less) in phosphate buffered saline. The ability of an antibody or antigen binding fragment to specifically bind a protein epitope can be determined using any appropriate method, such as binding to an immobilized substrate coupled to the target epitope with detection using an ELISA method, binding to the target epitope on live cells with detection by flow cytometry, or binding to an immobilized target epitope by surface plasmon resonance.

In some cases, an antibody that can be used to reduce the level of sPD-L1 can be an antibody that binds to inhibit both ADAM10 and ADAM17. One or more antibodies may have affinity to sequences on both ADAM10 and ADAM17, or may be combined, or a bispecific antibody can be used. Bispecific antibodies can be generated in an IgG-like structural format that includes two Fab arms and one Fc region like a typical antibody, except the two Fab sites bind different antigens, or in a non-IgG-like format that lacks an Fc region entirely. The latter format includes chemically linked Fabs that consist only of Fab regions, as well as various types of bivalent and trivalent scFvs. In some cases, fusion proteins can be generated to mimic the variable domains of two antibodies. Any appropriate method can be used to generate bispecific antibodies, including production via quadromas (e.g., hybrid hybridomas), chemical conjugation, and genetic recombination. For example, bispecific antibodies can be generated by genetically combining individual antibody coding sequences in a hybridoma.

Complete antibodies or portions of antibodies, bispecific antibodies, and similar molecules can be produced synthetically or semi-synthetically. In some cases, hybridoma technologies can be used to produce antibodies or portions thereof. B cells or plasma cells can be induced to produce antibodies or portions of antibodies through immunization of an animal (e.g., a mouse, rabbit, or another animal with a humoral immune response) with an antigen, followed by isolation of the desired cells. In some cases, artificial sequences can be introduced into B cells, plasma cells, or other suitable cell types. B cells or plasma cell lines producing the desired antibody or antibody portion can be fused with immortal B cell cancer cells, plasma cells, or myeloma cells to produce hybridomas. These hybridomas can reliably produce complete or partial antibody molecules that can be, for example, simple monoclonal antibodies, portions of bispecific antibodies, or complete bispecific antibodies. Progeny cells can be selected for their ability to survive, grow, and produce the desired products. In some cases, polyclonal antibodies can be generated with similar properties by inoculation of an animal with a humoral immune response. Bispecific antibodies or other combinations thus can be generated that inhibit ADAM10/ADAM17 as well as PD-1-PD-L1 interaction.

In some cases, PD-L1 inhibitors (e.g., anti-PD-L1 antibodies such as avelumab, atezolizumab, or durvalumab; and PD-1 receptor analogues such as rhPD1) also can be used to reduce the level of sPD-L1, thus reducing or removing the interference of sPD-L1 with the effectiveness of anti-PD-1 antibodies, anti-PD-L1 antibodies, and/or other inhibitors of PD-1/PD-L1 interactions, thereby increasing their effectiveness. The PD-L1 inhibitor may reduce levels of sPD-L1, which can have the downstream effect of (a) reducing or preventing sPD-L1 engagement with PD-1, thereby enhancing the effectiveness of an anti-PD-1 antibody or other inhibitor of PD-1/PD-L1 interactions, and/or (b) reducing or preventing sPD-L1 from binding to an administered anti-PD-L1 antibodies, thereby enhancing the effectiveness of the anti-PD-L1 antibody.

Any appropriate PD-L1 inhibitor can be used as described herein to reduce the level of sPD-L1 available to interact with PD-1 within a mammal (e.g., a human). For example, PD-L1 inhibitors can be used as described herein to reduce or prevent the accumulation of sPD-L1 from PD-L1 within a mammal (e.g., a human). In some cases, PD-L1 inhibitors can be used as described herein to reduce sPD-L1 within a mammal (e.g., a human). A PD-L1 inhibitor described herein can be a small molecule that inhibits interaction of sPD-L1 with PD-1, a decoy PD-1 receptor, or an antibody that binds to the sPD-L1 and inhibits its activity. Examples of PD-L1 inhibitors that can be used as described herein to reduce the level of sPD-L1 production within a mammal (e.g., a human) include, without limitation, anti-PD-L1 antibodies such as avelumab, atezolizumab, or durvalumab, and PD-1 polypeptides (e.g., recombinant PD-1 or a portion thereof that can bind to sPD-L1, or PD-1 receptor analogues such as rhPD1).

A representative full length amino acid sequence for human PD-1 is set forth in SEQ ID NO:7, and a representative nucleic acid sequence encoding human PD-1 is set forth in SEQ ID NO:8.

(SEQ ID NO: 7)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL
(SEQ ID NO: 8)
atgcagatcccacaggcgccctggccagtcgtctgggcggtgctacaact
gggctggcggccaggatggttcttagactccccagacaggccctggaacc
cccccaccttctccccagccctgctcgtggtgaccgaaggggacaacgcc
accttcacctgcagcttctccaacacatcggagagcttcgtgctaaactg
gtaccgcatgagccccagcaaccagacggacaagctggccgccttccccg
aggaccgcagccagcccggccaggactgccgcttccgtgtcacacaactg
cccaacgggcgtgacttccacatgagcgtggtcagggcccggcgcaatga
cagcggcacctacctctgtggggccatctccctggcccccaaggcgcaga
tcaaagagagcctgcgggcagagctcagggtgacagagagaagggcagaa
gtgcccacagcccaccccagcccctcacccaggccagccggccagttcca
aaccctggtggttggtgtcgtgggcggcctgctgggcagcctggtgctgc
tagtctgggtcctggccgtcatctgctcccgggccgcacgagggacaata
ggagccaggcgcaccggccagcccctgaaggaggacccctcagccgtgcc
tgtgttctctgtggactatggggagctggatttccagtggcgagagaaga
ccccggagccccccgtgccctgtgtccctgagcagacggagtatgccacc
attgtattcctagcggaatgggcacctcatcccccgcccgcaggggctca
gctgacggccctcggagtgcccagccactgaggcctgaggatggacactg
ctcttggcccctctga

A representative partial amino acid sequence for human PD-1 is set forth in SEQ ID NO:9, and is encoded by the representative nucleic acid sequence set forth in SEQ ID NO:10.

(SEQ ID NO: 9)
PGWFLDSPDRPWNPPTFSPALLVVTEGDNATFTCSFSNTSESFVLNWYRM
SPSNQTDKLAAFPEDRSQPGQDCRFRVTQLPNGRDFHMSVVRARRNDSGT
YLCGAISLAPKAQIKESLRAELRVTERRAEVPTAHPSPSPRPAGQFQTLV
(SEQ ID NO: 10)
ccaggatggttcttagactccccagacaggccctggaacccccccacctt
ctccccagccctgctcgtggtgaccgaaggggacaacgccaccttcacct
gcagcttctccaacacatcggagagcttcgtgctaaactggtaccgcatg
agccccagcaaccagacggacaagctggccgccttccccgaggaccgcag
ccagcccggccaggactgccgcttccgtgtcacacaactgcccaacgggc
gtgacttccacatgagcgtggtcagggcccggcgcaatgacagcggcacc
tacctctgtggggccatctccctggcccccaaggcgcagatcaaagagag
cctgcgggcagagctcagggtgacagagagaagggcagaagtgcccacag
cccaccccagcccctcacccaggccagccggccagttccaaaccctggtg

Any appropriate method can be used to generate a recombinant PD-1 polypeptide. For example, a nucleotide sequence encoding a PD-1 polypeptide (e.g., SEQ ID NO:8 or a portion thereof) can be inserted into an expression vector, which can be transfected into any appropriate cell such that the coding sequence can be expressed, and the resulting polypeptide can be isolated.

The term “polypeptide” as used herein refers to a compound of two or more subunit amino acids regardless of post-translational modification (e.g., phosphorylation or glycosylation). The subunits may be linked by peptide bonds or other bonds such as, for example, ester or ether bonds. The term “amino acid” refers to either natural and/or unnatural or synthetic amino acids, including D/L optical isomers.

By “isolated” or “purified” with respect to a polypeptide it is meant that the polypeptide is separated to some extent from the cellular components with which it is normally found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

The terms “nucleic acid” and “polynucleotide” are used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, “isolated,” when in reference to a nucleic acid, refers to a nucleic acid that is separated from other nucleic acids that are present in a genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be obtained by mutagenesis. For example, a donor nucleic acid sequence can be mutated using standard techniques, including oligonucleotide-directed mutagenesis and site-directed mutagenesis through PCR. See, Short Protocols in Molecular Biology, Chapter 8, Green Publishing Associates and John Wiley & Sons, Ausubel et al. (Ed.), 1992.

In some cases, a nucleic acid or polypeptide as described herein (e.g., an ADAM10 or ADAM17 polypeptide used to generate an antibody, or a PD-1 polypeptide used to inhibit PD-L1) can have a sequence that deviates from a reference sequence provided herein (e.g., SEQ ID NO:1, 2, 3, 4, 5, 6, 7, 8, 9, or 10). For example, an ADAM10 polypeptide sequence can have at least 80% sequence identity to SEQ ID NO:1 or an ADAM17 polypeptide sequence can have at least 80% sequence identity to SEQ ID NO:3 or SEQ ID NO:5, where the polypeptide includes one or more amino acid additions, subtractions, or substitutions compared to SEQ ID NO:1, SEQ ID NO:3, or SEQ ID NO:5. In some embodiments, a polypeptide sequence can have at least 85% sequence identity, 90% sequence identity, 95% sequence identity, 96% sequence identity, 97% sequence identity, 98% sequence identity, or 99% sequence identity to a reference sequence set forth herein, provided that it includes one or more amino acid additions, subtractions, or substitutions compared to the reference sequence.

Percent sequence identity is calculated by determining the number of matched positions in aligned nucleic acid or polypeptide sequences, dividing the number of matched positions by the total number of aligned nucleotides or amino acids, respectively, and multiplying by 100. A matched position refers to a position in which identical nucleotides or amino acids occur at the same position in aligned sequences. The total number of aligned nucleotides or amino acids refers to the minimum number of query (e.g., ADAM10, ADAM17, or PD-1) nucleotides or amino acids that are necessary to align the second sequence, and does not include alignment (e.g., forced alignment) with unrelated (e.g., non-ADAM10, non-ADAM17, or non-PD-1) sequences. The total number of aligned nucleotides or amino acids may correspond to the entire reference sequence or may correspond to fragments of the full-length reference sequences set forth herein.

Sequences can be aligned using the algorithm described by Altschul et al. (Nucleic Acids Res., 25:3389-3402, 1997) as incorporated into BLAST (basic local alignment search tool) programs, available at ncbi.nlm.nih.gov on the World Wide Web. BLAST searches or alignments can be performed to determine percent sequence identity between a query nucleic acid or amino acid molecule and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a query sequence and another sequence, the default parameters of the respective programs are used.

A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

In some cases, nucleic acids can include a “regulatory region” (also referred to as a “control element” or “expression control sequence”), which is a nucleotide sequence that influences transcription or translation initiation and rate, and/or stability or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, mitochondrial targeting sequences, and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in a cell in which it is desired to express a particular nucleic acid.

Recombinant nucleic acid constructs can include a polynucleotide sequence inserted into a vector suitable for transformation of cells (e.g., prokaryotic cells or eukaryotic cells, such as plant cells or animal cells). Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.). Vectors can be introduced into cells by any of a variety of methods (e.g., transfection, transformation, projectile bombardment, liposomes, or electroporation).

rhPD1 is a chimeric polypeptide that includes amino acids 25-167 of human PD-1 fused via a polypeptide linker (IEGRMD; SEQ ID NO:11) to the Fc portion of human IgG1. rhPD1 can be obtained commercially (e.g., from R&D Systems, Minneapolis, Minn.; catalog number 1086-PD-050).

Before, during, or after reducing or preventing the generation of sPD-L1 from PD-L1 within a mammal (e.g., a human) by administering a metallopeptidase inhibitor as described herein, or before, during, or after reducing the level of sPD-L1 available to interact with PD-1 within a mammal (e.g., a human) by administering a PD-L1 inhibitor as described herein, an inhibitor of PD-1/PD-L1 interactions can be administered to the mammal.

Any appropriate inhibitor of a PD-1/PD-L1 interaction can be used as described herein to reduce or prevent the effects of PD-L1 engagement of PD-1 within a mammal (e.g., a human). For example, anti-PD-1 antibodies, anti-PD-L1 antibodies, or combinations thereof can be used as described herein to reduce or prevent the effects of PD-L1 engagement of PD-1 within a mammal (e.g., a human). An anti-PD-1 antibody that can be used as described herein can be a polyclonal antibody, a monoclonal antibody a humanized antibody, a chimeric antibody, single chain Fv antibody fragment, a Fab fragment, or a F(ab)2 fragment that is capable of binding to an epitopic determinant of PD-1 (e.g., human PD-1). Examples of anti-PD1 antibodies that can be used as described herein include, without limitation, pembrolizumab (a humanized antibody with the trade name KEYTRUDA®, available from Merck), nivolumab (a targeted antibody with the trade name OPDIVO®, available from Bristol-Myers Squibb), and pidilizumab (a monoclonal antibody available from Medivation). An anti-PD-L1 antibody that can be used as described herein can be a polyclonal antibody, a monoclonal antibody a humanized antibody, a chimeric antibody, single chain Fv antibody fragment, a Fab fragment, or a F(ab)2 fragment that is capable of binding to an epitopic determinant of PD-L1 (e.g., human PD-L1). Examples of anti-PD-L1 antibodies that can be used as described herein include, without limitation, avelumab (a monoclonal antibody with the trade name BAVENCIO®, available from Pfizer), atezolizumab (a humanized monoclonal antibody with the trade name TECENTRIQ®, available from Genentech) and durvalumab (a monoclonal antibody with the trade IMFINZI®, available from AstraZeneca).

As described herein, this document provides materials and methods for enhancing the effectiveness of an inhibitor of PD-1/PD-L1 interactions (e.g., an anti-PD-1 antibody or an anti-PD-L1 antibody) using one or more metallopeptidase inhibitors (e.g., one or more MMP inhibitors, one or more ADAM10 inhibitors, one or more ADAM17 inhibitors, or a combination thereof) and one or more inhibitors of PD-1/PD-L1 interactions (e.g., one or more anti-PD-1 antibodies, one or more anti-PD-L1 antibodies, or a combination thereof). This document also provides materials and methods for enhancing the effectiveness of an inhibitor of PD-1/PD-L1 interactions (e.g., an anti-PD-1 antibody or an anti-PD-L1 antibody) using one or more PD-L1 inhibitors (e.g., anti-PD-L1 antibodies such as avelumab, atezolizumab, or durvalumab, and/or PD-1 receptor analogues such as rhPD1) and one or more inhibitors of PD-1/PD-L1 interactions (e.g., one or more anti-PD-1 antibodies, one or more anti-PD-L1 antibodies, or a combination thereof).

This document also provides methods for immunomodulatory treatment of cancer patients by administering a plurality of inhibitors to a patient, where the plurality of inhibitors includes one or more metallopeptidase inhibitors (e.g., one or more MMP inhibitors, one or more ADAM10 inhibitors, one or more ADAM17 inhibitors, or a combination thereof) and one or more inhibitors of PD-1/PD-L1 interactions (e.g., one or more anti-PD-1 antibodies, one or more anti-PD-L1 antibodies, or a combination thereof). In addition, this document provides methods for immunomodulatory treatment of cancer patients by administering a plurality of inhibitors to a patient, where the plurality of inhibitors includes one or more PD-L1 inhibitors (e.g., anti-PD-L1 antibodies such as avelumab, atezolizumab, or durvalumab, and/or PD-1 receptor analogues such as rhPD1) and one or more inhibitors of PD-1/PD-L1 interactions (e.g., one or more anti-PD-1 antibodies, one or more anti-PD-L1 antibodies, or a combination thereof).

In some embodiments, this document provides a system for immunomodulatory treatment of a cancer patient, where the system includes manufactured combinatorial therapeutics that contain (a) one or more metallopeptidase inhibitors (e.g., one or more MMP inhibitors, one or more ADAM10 inhibitors, one or more ADAM17 inhibitors, or a combination thereof) and (b) one or more inhibitors of PD-1/PD-L1 interactions (e.g., one or more anti-PD-1 antibodies, one or more anti-PD-L1 antibodies, or a combination thereof), where the therapeutics in combination enhance the immune system's killing of tumor cells. In still other embodiments, this document provides a system for immunomodulatory treatment of a cancer patient, where the system includes manufactured combinatorial therapeutics that contain (a) one or more PD-L1 inhibitors (e.g., anti-PD-L1 antibodies such as avelumab, atezolizumab, or durvalumab, and/or PD-1 receptor analogues such as rhPD1) and (b) one or more inhibitors of PD-1/PD-L1 interactions (e.g., one or more anti-PD-1 antibodies, one or more anti-PD-L1 antibodies, or a combination thereof), where the therapeutics in combination enhance the immune system's killing of tumor cells.

The materials and methods provided herein can be used to modulate the effectiveness of inhibitors of PD-1/PD-L1 interaction (e.g., one or more anti-PD-1 antibodies, one or more anti-PD-L1 antibodies, or a combination thereof) through the use of one or more metallopeptidase inhibitors (e.g., one or more MMP inhibitors, one or more ADAM10 inhibitors, one or more ADAM17 inhibitors, or a combination thereof), or through the use of one or more PD-L1 inhibitors (e.g., anti-PD-L1 antibodies such as avelumab, atezolizumab, or durvalumab, and/or PD-1 receptor analogues such as rhPD1). For example, the materials and methods provided herein can inhibit sPD-L1 production or reduce the level of sPD-L1 available to interact with PD-1, to prevent downregulation of the immune system, thus enhancing the effectiveness of inhibitors of PD-1/PD-L1 interactions. The materials and methods can be used to, for example, increase healthy immune cell survival, kill tumor cells, and/or reduce tumor size.

In some embodiments, the methods provided herein can include administering (a) one or more metallopeptidase inhibitors (e.g., one or more MMP inhibitors, one or more ADAM10 inhibitors, one or more ADAM17 inhibitors, or a combination thereof and (b) one or more inhibitors of PD-1/PD-L1 interactions (e.g., one or more anti-PD-1 antibodies, one or more anti-PD-L1 antibodies, or a combination thereof) to a mammal (e.g., a human, non-human primate, horse, cow, pig, sheep, goat, cat, rabbit, guinea pig, hamster, rat, gerbil, or mouse) identified as being in need thereof. In some cases, the mammal can have a cancer, such as melanoma (e.g., metastatic melanoma), renal cancer, lung cancer (e.g., non-small cell lung cancer; NSCLC), mesothelioma, squamous cell cancer, a hematological cancer (e.g., leukemia or lymphoma, such as Hodgkin's lymphoma), neurological cancer, breast cancer, prostate cancer, head and neck cancer, gastrointestinal cancer, liver cancer, pancreatic cancer, genitourinary cancer, bone cancer, bladder cancer, or vascular cancer.

In some embodiments, the methods provided herein can include administering (a) one or more PD-L1 inhibitors (e.g., one or more anti-PD-L1 antibodies such as avelumab, atezolizumab, or durvalumab, and/or PD-1 receptor analogues such as rhPD1) and (b) one or more inhibitors of PD-1/PD-L1 interactions (e.g., one or more anti-PD-1 antibodies, one or more anti-PD-L1 antibodies, or a combination thereof) to a mammal (e.g., a human, non-human primate, horse, cow, pig, sheep, goat, cat, rabbit, guinea pig, hamster, rat, gerbil, or mouse) identified as being in need thereof. In some cases, the mammal can have a cancer, such as melanoma (e.g., metastatic melanoma), renal cancer, lung cancer (e.g., non-small cell lung cancer; NSCLC), mesothelioma, squamous cell cancer, a hematological cancer (e.g., leukemia or lymphoma, such as Hodgkin's lymphoma), neurological cancer, breast cancer, prostate cancer, head and neck cancer, gastrointestinal cancer, liver cancer, pancreatic cancer, genitourinary cancer, bone cancer, bladder cancer, or vascular cancer.

A subject that is anti-PD-1 resistant or anti-PD-L1 resistant may not respond to inhibitors of PD-1/PD-L1 interactions in an effective manner. In the methods provided herein, the subject in need of treatment can be a mammal identified as being anti-PD-1 resistant or anti-PD-L1 resistant; in some cases, the methods provided herein can include identifying a subject as being anti-PD-1 resistant or anti-PD-L1 resistant. A subject in need of the methods provided herein can be identified based on, for example, detection of sPD-L1 expression in a biological fluid sample (e.g., blood, plasma, serum, or urine), measurement of an elevated level of sPD-L1 expression in a biological fluid sample, detection of a reduced level of PD-L1 in a biological sample containing tumor cells, detection of ADAM10 and/or ADAM17 in a biological sample containing tumor cells, detection of an elevated level of ADAM10 and/or ADAM17 in a biological sample containing tumor cells, or a combination of methods that include assessing the presence or level of sPD-L1, the presence or level of PD-L1, the presence or level of ADAM10 and/or ADAM17, or any combination thereof. Having the ability to identify mammals as having a tumor that is resistant to treatment with anti-PD-1 or anti-PD-L1 antibodies can allow those mammals to be properly identified and treated in an effective and reliable manner. For example, the cancer treatments provided herein (e.g., one or more ADAM10 and/or ADAM17 inhibitors, one or more PD-L1 inhibitors, and one or more inhibitors of PD-1/PD-L1 interactions) can be used to treat patients identified as having a tumor resistant to inhibitors of PD-1/PD-L1 interactions. Thus, the methods provided herein can be used to determine which patients are more likely to benefit from checkpoint inhibitor treatment alone, and which patients are more likely to require additional treatment to reduce sPD-L1 levels or availability, in addition to treatment with a checkpoint inhibitor.

An elevated level of sPD-L1 is any level that is greater than a corresponding reference level for sPD-L1, where the reference level of sPD-L1 is the level of sPD-L1 typically found in mammals free of cancer. An elevated level of sPD-L1 can be, for example, 3 to 5% greater, 5 to 10% greater, 10 to 20% greater, 20 to 50% greater, 50 to 100% greater, or more than 100% greater than a reference level of sPD-L1. In some cases, an elevated level of sPD-L1 can be a level that is at least 2 percent (e.g., at least 3, 4, 5, 6, 7, 8, 9, 10, 15, 20, 25, 30, 35, 40, 45, 50, 55, 60, 65, 70, 75, 80, 85, 90, 95, 100, 200, 300, 400, or 500 percent) greater than a corresponding reference level. In addition, a reference level can be any amount. For example, a reference level for sPD-L1 can be zero. In this case, any level of sPD-L1 greater than zero can be considered an elevated level.

A reference level of sPD-L1 for a mammal can be the median level of sPD-L1 that is present in samples obtained from a random sampling of mammals of the same species that are free of cancer. Control samples used to determine a reference level can be obtained from any appropriate number of mammals (e.g., 10, 20, 30, 40, 50, 75, 100, 125, 150, 175, 200, 250, 300, 400, 500, 600, 700, 800, 900, 1000 or more mammals) from the same species as the mammal being evaluated. In some cases, when the mammal is a human, control samples can be samples from humans of the same race, age group, and/or geographic location as the human being evaluated.

A reduced level of PD-L1 is any level that is less than a corresponding reference level for PD-L1, where the reference level of PD-L1 is the level of PD-L1 typically expressed in mammals free of cancer. A reduced level of PD-L1 can be, for example, 3 to 5% less, 5 to 10% less, 10 to 20% less, 20 to 50% less, or 50 to 100% less than a reference level of PD-L1. In some cases, a reduced level of PD-L1 can be a level that is at least 2 percent (e.g., at least 3, 4, 5, 6, 7, 8, 9, 10, 15, 20, 25, 30, 35, 40, 45, 50, 55, 60, 65, 70, 75, 80, 85, 90, 95, or 100 percent) less than a corresponding reference level. In addition, a reference level can be any amount.

A reference level of PD-L1 for a mammal can be the median level of PD-L1 that is present in samples obtained from a random sampling of mammals of the same species that are free of cancer. Control samples used to determine a reference level can be obtained from any appropriate number of mammals (e.g., 10, 20, 30, 40, 50, 75, 100, 125, 150, 175, 200, 250, 300, 400, 500, 600, 700, 800, 900, 1000 or more mammals) from the same species as the mammal being evaluated. In some cases, when the mammal is a human, control samples can be samples from humans of the same race, age group, and/or geographic location as the human being evaluated.

An elevated level of ADAM10 or ADAM17 is any level that is greater than a corresponding reference level for ADAM10 or ADAM17, where the reference level of ADAM10 or ADAM17 is the level of ADAM10 or ADAM17 typically expressed in mammals free of cancer. An elevated level of ADAM10 or ADAM17 can be, for example, 3 to 5% greater, 5 to 10% greater, 10 to 20% greater, 20 to 50% greater, 50 to 100% greater, or more than 100% greater than a reference level of ADAM10 or ADAM17. In some cases, an elevated level of ADAM10 or ADAM17 can be a level that is at least 2 percent (e.g., at least 3, 4, 5, 6, 7, 8, 9, 10, 15, 20, 25, 30, 35, 40, 45, 50, 55, 60, 65, 70, 75, 80, 85, 90, 95, 100, 200, 300, 400, or 500 percent) greater than a corresponding reference level. In addition, a reference level can be any amount. For example, a reference level for ADAM10 or ADAM17 can be zero. In this case, any level of ADAM10 or ADAM17 greater than zero can be considered an elevated level.

A reference level of ADAM10 or ADAM17 for a mammal can be the median level of ADAM10 or ADAM17 that is present in samples obtained from a random sampling of mammals of the same species that are free of cancer. Control samples used to determine a reference level can be obtained from any appropriate number of mammals (e.g., 10, 20, 30, 40, 50, 75, 100, 125, 150, 175, 200, 250, 300, 400, 500, 600, 700, 800, 900, 1000 or more mammals) from the same species as the mammal being evaluated. In some cases, when the mammal is a human, control samples can be samples from humans of the same race, age group, and/or geographic location as the human being evaluated.

It will be appreciated that levels from comparable samples are used when determining whether or not a particular level of sPD-L1, PD-L1, ADAM10, or ADAM17 is an elevated or reduced level. For example, the median level of sPD-L1 present in serum from a random sampling of mammals may be X units/g of serum, while the median level of sPD-L1 present in urine may be Y units/g of urine. In this case, the reference level for sPD-L1 in serum would be X units/g of serum, and the reference level for sPD-L1 in urine would be Y units/g of urine. Thus, when determining whether or not the level of sPD-L1 in serum is elevated, the measured level would be compared to the reference level in serum. In addition, a level of sPD-L1 in a body fluid from a mammal typically is compared to a reference level determined by analyzing samples using a technique comparable to the technique used to measure the sPD-L1 level in the mammal being evaluated.

Methods for detecting and/or quantifying sPD-L1 in body fluids can include, for example, immunological techniques. For example, an antibody that binds to an epitope specific for sPD-L1 can be used to detect sPD-L1 in body fluid. In some cases, an antibody directed against sPD-L1 can bind the polypeptide with an affinity of at least 10⁻⁴ M (e.g., at least 10⁻⁵, 10⁻⁶, 10⁻⁷, 10⁻⁸, 10⁻⁹, 10⁻¹⁰, 10⁻¹¹, or 10⁻¹² M).

Antibodies having specific binding affinity for sPD-L1 can be commercially obtained, or can be produced using, for example, methods described elsewhere (see, for example, Dong et al., Nature Med 8:793-800, 2002). In some cases, a sPD-L1 polypeptide (e.g., a polypeptide comprising or consisting of the extracellular domain of PD-L1) can be recombinantly produced, or can be purified from a biological sample, and used to immunize a host animal such as, without limitation, a rabbit, chicken, mouse, guinea pig, or rat. Various adjuvants that can be used to increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin and dinitrophenol. Monoclonal antibodies can be prepared using a sPD-L1 polypeptide and hybridoma technology.

In immunological assays, an antibody having specific binding affinity for sPD-L1 or a secondary antibody that binds to such an antibody can be labeled, either directly or indirectly. Suitable labels include, without limitation, radioisotopes (e.g., ¹²⁵I, ¹³¹I, ³⁵S, ³H, ³²P, ³³P, or ¹⁴C), fluorophores (e.g., fluorescein, fluorescein-5-isothiocyanate (FITC), PerCP, rhodamine, or phycoerythrin), luminescent moieties (e.g., QDOT™ nanoparticles supplied by the Quantum Dot Corporation, Palo Alto, Calif.), compounds that absorb light of a defined wavelength, or enzymes (e.g., alkaline phosphatase or horseradish peroxidase). In some cases, antibodies can be indirectly labeled by conjugation with biotin and then detected with avidin or streptavidin labeled with a molecule described above. Methods of detecting or quantifying a label depend on the nature of the label, and can include, for example, the use of detectors such as x-ray film, radioactivity counters, scintillation counters, spectrophotometers, colorimeters, fluorometers, luminometers, and densitometers. Combinations of these approaches (including “multi-layer” assays) can be used to enhance the sensitivity of an assay.

Immunological assays for detecting sPD-L1 can be performed in a variety of formats, including sandwich assays (e.g., ELISA assays, sandwich Western blotting assays, or sandwich immunomagnetic detection assays), competition assays (competitive RIA), or bridge immunoassays. See, for example, U.S. Pat. Nos. 5,296,347; 4,233,402; 4,098,876; and 4,034,074. Methods of detecting sPD-L1 generally can include contacting a body fluid with an antibody that binds to sPD-L1 and detecting or quantifying binding of sPD-L1 to the antibody. For example, an antibody having specific binding affinity for sPD-L1 can be immobilized on a solid substrate and then exposed to the biological sample. In some cases, binding of sPD-L1 to the antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB; Rapsgatan, Sweden). Alternatively, the antibody can be labeled and detected as described above. A standard curve using known quantities of sPD-L1 can be generated to aid in the quantitation of sPD-L1 levels.

In some embodiments, a “sandwich” assay in which a capture antibody or capture binding substrate is immobilized on a solid substrate can be used to detect the presence, absence, or amount of sPD-L1. The solid substrate can be contacted with the biological sample such that sPD-L1 in the sample can bind to the immobilized antibody. The presence of sPD-L1 bound to the antibody can be determined using a “reporter” antibody having specific binding affinity for sPD-L1 and the methods described above. It is understood that in these sandwich assays, the capture antibody or capture binding substrate (e.g., an immobilized PD-1 receptor fragment) should not bind to the same epitope (or range of epitopes in the case of a polyclonal antibody) as the reporter antibody. Thus, if a monoclonal antibody is used as a capture antibody, the reporter antibody can be another monoclonal antibody that binds to an epitope that is either completely physically separated from or only partially overlaps with the epitope to which the capture monoclonal antibody binds, or a polyclonal antibody that binds to epitopes other than or in addition to that to which the capture monoclonal antibody binds.

Suitable solid substrates to which an antibody (e.g., a capture antibody) or capture binding substrate can be bound include, without limitation, microtiter plates, tubes, membranes such as nylon or nitrocellulose membranes, and beads or particles (e.g., agarose, cellulose, glass, polystyrene, polyacrylamide, magnetic, or magnetizable beads or particles). Magnetic or magnetizable particles can be used when an automated immunoassay system is used.

Alternative techniques for detecting sPD-L1 include mass-spectrophotometric techniques such as electrospray ionization (ESI), liquid chromatography-mass spectrometry (LC-MS), and matrix-assisted laser desorption-ionization (MALDI). See, for example, Gevaert et al., Electrophoresis, 22(9):1645-51, 2001; and Chaurand et al., J Am Soc Mass Spectrom, 10(2):91-103, 1999). Mass spectrometers useful for such applications are available from Applied Biosystems (Foster City, Calif.); Bruker Daltronics (Billerica, Mass.) and Amersham Pharmacia (Sunnyvale, Calif.). Arrays for detecting polypeptides, two-dimensional gel analysis, and chromatographic separation techniques also can be used to detect sPD-L1.

Methods for detecting and/or quantifying PD-L1, ADAM10, and/or ADAM17 can include, for example, immunological techniques. For example, an antibody that binds to an epitope specific for PD-L1, ADAM10, or ADAM17 can be used to detect PD-L1, ADAM10, or ADAM17 in a biological sample (e.g., a tumor sample). In some cases, an antibody directed against PD-L1, ADAM10, or ADAM17 can bind to PD-L1, ADAM10, or ADAM17, respectively, with an affinity of at least 10⁻⁴ M (e.g., at least 10⁻⁵, 10⁻⁶, 10⁻⁷, 10⁻⁸, 10⁻⁹, 10⁻¹⁰, 10⁻¹¹, or 10⁻¹² M).

Antibodies having specific binding affinity for PD-L1, ADAM10, or ADAM17 can be commercially obtained, or can be produced using, for example, methods described elsewhere (see, for example, Dong et al., supra). In some cases, a PD-L1, ADAM10, or ADAM17 polypeptide can be recombinantly produced, or can be purified from a biological sample, and used to immunize a host animal such as, without limitation, a rabbit, chicken, mouse, guinea pig, or rat. Various adjuvants that can be used to increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin and dinitrophenol. Monoclonal antibodies can be prepared using a ADAM10 or ADAM17 polypeptide (e.g., a fragment of SEQ ID NO:1, SEQ ID NO:3, or SEQ ID NO:5) and hybridoma technology, for example.

In immunological assays, an antibody having specific binding affinity for PD-L1, ADAM10, or ADAM17 or a secondary antibody that binds to such an antibody can be labeled, either directly or indirectly. Suitable labels include, without limitation, radioisotopes (e.g., ¹²⁵I, ¹³¹I, ³⁵S, ³H, ³²P, ³³P, or ¹⁴C), fluorophores (e.g., fluorescein, fluorescein-5-isothiocyanate (FITC), PerCP, rhodamine, or phycoerythrin), luminescent moieties (e.g., QDOT™ nanoparticles supplied by the Quantum Dot Corporation), compounds that absorb light of a defined wavelength, or enzymes (e.g., alkaline phosphatase or horseradish peroxidase). In some cases, antibodies can be indirectly labeled by conjugation with biotin and then detected with avidin or streptavidin labeled with a molecule described above. Methods of detecting or quantifying a label depend on the nature of the label, and can include, for example, the use of detectors such as x-ray film, radioactivity counters, scintillation counters, spectrophotometers, colorimeters, fluorometers, luminometers, and densitometers. Combinations of these approaches (including “multi-layer” assays) can be used to enhance the sensitivity of an assay.

Immunological assays for detecting PD-L1, ADAM10, or ADAM17 can be performed in a variety of formats, including sandwich assays (e.g., ELISA assays, sandwich Western blotting assays, or sandwich immunomagnetic detection assays), competition assays (competitive RIA), or bridge immunoassays. See, for example, U.S. Pat. Nos. 5,296,347; 4,233,402; 4,098,876; and 4,034,074. In general, methods of detecting PD-L1, ADAM10, or ADAM17 can include contacting a biological sample with an antibody that binds to PD-L1, ADAM10, or ADAM17, or with a combination of antibodies that bind to two or more of PD-L1, ADAM10, and ADAM17, and detecting or quantifying binding of the antibodies to PD-L1, ADAM10, and/or ADAM17. In some cases, antibodies can be labeled and detected as described above. A standard curve using known quantities of PD-L1, ADAM10, and/or ADAM17 can be generated to aid in the quantitation of PD-L1, ADAM10, and/or ADAM17 levels.

In some cases, two or more of these markers can be evaluated, either separately or simultaneously (e.g., in a single assay). For example, the presence or level of ADAM10 and ADAM17, ADAM10 and PD-L1, ADAM17 and PD-L1, or ADAM10, ADAM17, and PD-L1 can be assessed at the same time, in the same assay sample. In some cases, multiplex ADAM10/ADAM17/PD-L1 immunofluorescence (IF) can be used to evaluate the presence or level of all three polypeptides in a single assay (e.g., in a single sample from a patient), to predict the likelihood that a patient will respond to checkpoint inhibitor therapy. In addition, the presence or level of sPD-L1 in a body fluid sample (e.g., blood, serum, plasma, or urine) from the patient also can be assessed in conjunction with any of the aforementioned combinations of assays. The presence of an elevated level of ADAM10 and/or ADAM17, a reduced level of PD-L1, or both an elevated level of ADAM10 and/or ADAM17 and a reduced level of PD-L1 in a sample (e.g., a tumor sample) from a patient can indicate that the patient is likely to be resistant to treatment with an inhibitor of PD-1/PD-L1 interactions, and also can indicate that the patient is likely to benefit from treatment with (1) one or more metallopeptidase inhibitors (e.g., one or more MMP inhibitors, one or more ADAM10 inhibitors, one or more ADAM17 inhibitors, or a combination thereof) or one or more PD-L1 inhibitors (e.g., anti-PD-L1 antibodies such as avelumab, atezolizumab, or durvalumab, and/or PD-1 receptor analogues such as rhPD1), and (2) one or more inhibitors of PD-1/PD-L1 interactions (e.g., one or more anti-PD-1 antibodies, one or more anti-PD-L1 antibodies, or a combination thereof). The detection of sPD-L1 in a body fluid sample from the patient, or the determination that the level of sPD-L1 is elevated in a body fluid sample from the patient, particularly in combination with the determination of an elevated level of ADAM10 and/or ADAM17, a reduced level of PD-L1, or both an elevated level of ADAM10 and/or ADAM17 and a reduced level of PD-L1 in a sample from the patient, can bolster the indications that the patient is likely to be resistant to treatment with an inhibitor of PD-1/PD-L1 interactions, and that the patient is likely to benefit from treatment with (1) one or more metallopeptidase inhibitors or one or more PD-L1 inhibitors and (2) one or more inhibitors of PD-1/PD-L1 interactions. Any appropriate method can be used for simultaneous detection or quantification of ADAM10, ADAM17, PD-L1, or any combination thereof, including CODEX multiplex IF technology (Akoya Biosciences; Menlo Park, Calif.).

A subject identified as being in need of the methods provided herein can be administered (a) one or more metallopeptidase inhibitors (e.g., one or more MMP inhibitors, one or more ADAM10 inhibitors, one or more ADAM17 inhibitors, or a combination thereof) or one or more PD-L1 inhibitors (e.g., one or more anti-PD-L1 antibodies, on or more PD-1 analogues, or a combination thereof), and (b) one or more inhibitors of PD-1/PD-L1 interactions (e.g., one or more anti-PD-1 antibodies, one or more anti-PD-L1 antibodies, or a combination thereof).

The one or more metallopeptidase inhibitors and the one or more inhibitors of PD-1/PD-L1 interactions can be administered simultaneously or separately (e.g., sequentially) to a subject. For example, one or more metalloprotease inhibitors can be combined with one or more anti-PD-1 antibodies or one or more anti-PD-L1 antibodies in a single composition for administration to a subject in need thereof. Alternatively, one or more metallopeptidase inhibitors (e.g., one or more MMP inhibitors, one or more ADAM10 inhibitors, one or more ADAM17 inhibitors, or a combination thereof) can be administered to a subject, and one or more inhibitors of PD-1/PD-L1 interactions (e.g., one or more anti-PD-1 antibodies, one or more anti-PD-L1 antibodies, or a combination thereof) can subsequently be administered at a later time point (e.g., 30 to 60 minutes, 1 to 4 hours, 4 to 12 hours, 12 to 24 hours, 1 to 3 days, 3 to 7 days, or more than 7 days later). For example, one or more MMP inhibitors can be administered to a subject in need thereof, to reduce the level of sPD-L1 in the subject, and then one or more inhibitors of PD-1/PD-L1 interactions can be administered to the subject. In some cases, more than one dose (e.g., two, three, four, or more than four doses) of either or both types of inhibitors can be administered.

The one or more PD-L1 inhibitors and the one or more inhibitors of PD-1/PD-L1 interactions can be administered simultaneously or separately (e.g., sequentially) to a subject. For example, one or more PD-L1 inhibitors can be combined with one or more anti-PD-1 antibodies in a single composition for administration to a subject in need thereof. Alternatively, one or more PD-L1 inhibitors (e.g., one or more anti-PD-L1 antibodies, on or more PD-1 analogues, or a combination thereof) can be administered to a subject, and one or more inhibitors of PD-1/PD-L1 interactions (e.g., one or more anti-PD-1 antibodies, one or more anti-PD-L1 antibodies, or a combination thereof) can subsequently be administered at a later time point (e.g., 30 to 60 minutes, 1 to 4 hours, 4 to 12 hours, 12 to 24 hours, 1 to 3 days, 3 to 7 days, or more than 7 days later). For example, one or more PD-L1 inhibitors can be administered to a subject in need thereof, to reduce the level of sPD-L1 in the subject, and then one or more inhibitors of PD-1/PD-L1 interactions can be administered to the subject. In some cases, more than one dose (e.g., two, three, four, or more than four doses) of either or both types of inhibitors can be administered.

One or more metallopeptidase inhibitors or PD-L1 inhibitors described herein and one or more inhibitors of PD-1/PD-L1 interactions described herein can be incorporated into compositions for administration to a subject (e.g., human or a non-human mammal with cancer). Any appropriate method can be used to formulate and subsequently administer a therapeutic composition provided herein. For example, one or more metallopeptidase inhibitors and one or more inhibitors of PD-1/PD-L1 interactions can be admixed, encapsulated, conjugated or otherwise associated with other molecules, molecular structures, or mixtures of compounds such as, for example, liposomes, receptor or cell targeted molecules, or oral, topical or other formulations for assisting in uptake, distribution and/or absorption.

In some embodiments, a composition can contain one or more metallopeptidase inhibitors (e.g., one or more MMP inhibitors, one or more ADAM10 inhibitors, one or more ADAM17 inhibitors, or a combination thereof) and one or more inhibitors of PD-1/PD-L1 interactions (e.g., one or more anti-PD-1 antibodies, one or more anti-PD-L1 antibodies, or a combination thereof) in combination with a pharmaceutically acceptable carrier. Pharmaceutically acceptable carriers include, for example, pharmaceutically acceptable solvents, suspending agents, or any other pharmacologically inert vehicles for delivering, e.g., antibodies to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water; saline solution; binding agents (e.g., polyvinylpyrrolidone or hydroxypropyl methylcellulose); fillers (e.g., lactose or dextrose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions can be administered by a number of methods, depending upon whether local or systemic treatment is desired. Administration can be, for example, parenteral (e.g., by subcutaneous, intrathecal, intraventricular, intramuscular, or intraperitoneal injection, or by intravenous (i.v.) drip); oral; topical (e.g., transdermal, sublingual, ophthalmic, or intranasal); or pulmonary (e.g., by inhalation or insufflation of powders or aerosols), or can occur by a combination of such methods. Administration can be rapid (e.g., by injection) or can occur over a period of time (e.g., by slow infusion or administration of slow release formulations).

Compositions and formulations for parenteral, intrathecal or intraventricular administration include sterile aqueous solutions (e.g., sterile physiological saline), which also can contain buffers, diluents and other suitable additives (e.g., penetration enhancers, carrier compounds and other pharmaceutically acceptable carriers).

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Pharmaceutical compositions include, but are not limited to, solutions, emulsions, aqueous suspensions, and liposome-containing formulations. The compositions can be generated from a variety of components that include, for example, preformed liquids, self-emulsifying solids and self-emulsifying semisolids. Emulsion formulations can be particularly useful for oral delivery of therapeutic compositions due to their ease of formulation and efficacy of solubilization, absorption, and bioavailability. Liposomes can be particularly useful due to their specificity and the duration of action they offer from the standpoint of drug delivery.

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions, such as dyes, flavoring agents, preservatives, antioxidants, opacifiers, thickening agents, and stabilizers. Further, a composition can be mixed with auxiliary agents, e.g., lubricants, preservatives, stabilizers, wetting agents, emulsifiers, salts for influencing osmotic pressure, buffers, colorings, flavorings, penetration enhancers, and aromatic substances. When added, however, such materials should not unduly interfere with the biological activities of the other components within the compositions.

Pharmaceutical formulations as disclosed herein, which can be presented conveniently in unit dosage form, can be prepared according to conventional techniques well known in the pharmaceutical industry. Such techniques include the step of bringing into association the active ingredients (e.g., antibodies) with the desired pharmaceutical carrier(s). Typically, the formulations can be prepared by uniformly and intimately bringing the active ingredients into association with liquid carriers or finely divided solid carriers or both, and then, if necessary, shaping the product. Formulations can be sterilized if desired, provided that the method of sterilization does not interfere with the effectiveness of the molecules(s) contained in the formulation.

Dosages typically are dependent on the responsiveness of the subject to the therapies, with the course of treatment lasting from several days to several months, or until a suitable response is achieved. Any appropriate method can be used to determine optimum dosages, dosing methodologies and repetition rates. Optimum dosages can vary depending on the relative potency of an antibody, and generally can be estimated based on the EC₅₀ found to be effective in in vitro and/or in vivo animal models. Compositions containing the therapeutics can be given once or more daily, weekly, monthly, or even less often, or can be administered continuously for a period of time (e.g., hours, days, or weeks).

In some embodiments, a composition containing one or more metallopeptidase inhibitors, one or more PD-L1 inhibitors (e.g., rhPD1 or an anti-PD-L1 antibody) and/or one or more inhibitors of PD-1/PD-L1 interaction can be administered such that each of the one or more inhibitors in the composition is administered at a dose of at least about 0.01 ng/kg to about 100 mg/kg of body mass (e.g., about 10 ng/kg to about 50 mg/kg, about 20 ng/kg to about 10 mg/kg, about 0.1 ng/kg to about 20 ng/kg, about 3 ng/kg to about 10 ng/kg, or about 50 ng/kg to about 100 μg/kg) of body mass, although other dosages also may provide beneficial results. It is to be noted that the dosage of each therapeutic in a composition can be independent of the dosages of the other therapeutics in the composition, such that they are administered at different amounts.

In some cases, one or more metallopeptidase inhibitors and/or one or more inhibitors of PD-1/PD-L1 interaction can be administered once or more than once (e.g., by repeated injections, oral administrations, or by use of a series of transdermal drug patches). When administered more than once, the frequency of administration can range from about four times a day to about once every other month (e.g., twice a day, once a day, three to five times a week, about once a week, about twice a month, about once a month, or about once every other month). In addition, the frequency of administration can remain constant or can be variable during the duration of treatment. Various factors can influence the actual frequency of administration used for a particular application. For example, the effective amount, duration of treatment, route of administration, and severity of condition may require an increase or decrease in administration frequency.

A composition containing one or more metallopeptidase inhibitors (e.g., one or more MMP inhibitors, one or more ADAM10 inhibitors, one or more ADAM17 inhibitors, or a combination thereof) and/or one or more inhibitors of PD-1/PD-L1 interaction (e.g., one or more anti-PD-1 antibodies, one or more anti-PD-L1 antibodies, or a combination thereof) can be administered to a subject in an amount, at a frequency, and for a duration effective to achieve a desired effect (e.g., to reduce tumor size, reduce cancer cell number, to reduce one or more symptoms of cancer, or to prevent or delay worsening of one or more such symptoms). For example, the one or more metallopeptidase inhibitors can be administered in an amount effective to reduce the level of sPD-L1, reduce or prevent production and/or secretion of sPD-L1, or to reduce or prevent sPD-L1 from engaging with its counterpart receptors. “Reduced expression,” “reduced secretion,” and “reduced production” of sPD-L1 are levels of expression, secretion, and production, respectively, that are at least 3% (e.g., at least 5%, at least 10%, at least 20%, at least 50%, 3% to 5%, 5% to 10%, 10% to 15%, 15% to 20%, 20% to 25%, 25% to 50%, or more than 50%) lower than a previously determined level of sPD-L1 expression, secretion, or production, based on the amounts of sPD-L1 mRNA or protein in samples obtained from a subject before, during, and/or after treatment. In combination, one or more metallopeptidase inhibitors and one or more inhibitors of PD-1/PD-L1 interaction can be administered in amounts effective to reduce the size of a tumor, reduce the number of cancer cells, or reduce one or more symptoms of cancer in a patient by at least 3% (e.g., at least 5%, at least 10%, at least 20%, at least 50%, 3% to 5%, 5% to 10%, 10% to 15%, 15% to 20%, 20% to 25%, 25% to 50%, or more than 50%). In some cases, for example, effective amounts of metallopeptidase inhibitors and inhibitors of PD-1/PD-L1 interactions described herein can be amounts that reduce the size of a tumor in treated mammal by at least 10% as compared to the size of the tumor in the mammal prior to administration of the inhibitors. The presence or extent of tumors, cancer cells, and cancer symptoms can be evaluated using any appropriate method.

A composition containing one or more PD-L1 inhibitors (e.g., one or more anti-PD-L1 antibodies, one or more PD-1 analogues, or a combination thereof) and/or one or more inhibitors of PD-1/PD-L1 interaction (e.g., one or more anti-PD-1 antibodies, one or more anti-PD-L1 antibodies, or a combination thereof) can be administered to a subject in an amount, at a frequency, and for a duration effective to achieve a desired effect (e.g., to reduce tumor size, reduce cancer cell number, to reduce one or more symptoms of cancer, or to prevent or delay worsening of one or more such symptoms). For example, the one or more PD-L1 inhibitors can be administered in an amount effective to reduce the level of available sPD-L1 or to reduce or prevent sPD-L1 from engaging with its counterpart receptors. A “reduced level” of sPD-L1 is a levels that is at least 3% (e.g., at least 5%, at least 10%, at least 20%, at least 50%, 3% to 5%, 5% to 10%, 10% to 15%, 15% to 20%, 20% to 25%, 25% to 50%, or more than 50%) lower than a previously determined level of sPD-L1, based on the amount of sPD-L1 protein in samples obtained from a subject before, during, and/or after treatment as measured by, for example, flow cytometry, ELISA, Western analysis, or any other appropriate method. In combination, one or more PD-L1 inhibitors and one or more inhibitors of PD-1/PD-L1 interaction can be administered in amounts effective to reduce the size of a tumor, reduce the number of cancer cells, or reduce one or more symptoms of cancer in a patient by at least 3% (e.g., at least 5%, at least 10%, at least 20%, at least 50%, 3% to 5%, 5% to 10%, 10% to 15%, 15% to 20%, 20% to 25%, 25% to 50%, or more than 50%). In some cases, for example, effective amounts of PD-L1 inhibitors and inhibitors of PD-1/PD-L1 interactions described herein can be amounts that reduce the size of a tumor in treated mammal by at least 10% as compared to the size of the tumor in the mammal prior to administration of the inhibitors. The presence or extent of tumors, cancer cells, and cancer symptoms can be evaluated using any appropriate method.

In some embodiments, the amounts of one or more metallopeptidase inhibitors and one or more inhibitors of PD-1/PD-L1 interactions administered to a mammal and/or the frequency of administration can be titrated in order to, for example, identify a dosage that is most effective to treat the mammal while having the least amount of adverse effects. For example, an effective amount of a composition containing both one or more metallopeptidase inhibitors and one or more inhibitors of PD-1/PD-L1 interaction can be any amount that reduces tumor size or reduces cancer symptoms within a mammal, without having significant toxicity in the mammal. If a mammal fails to respond to a particular amount, then the amount can be increased by, for example, two-fold, three-fold, five-fold, or ten-fold. After receiving this higher concentration, the mammal can be monitored for both responsiveness to the treatment and toxicity symptoms, and adjustments in the dosage can be made accordingly. The effective amount can remain constant or can be adjusted as a sliding scale or variable dose depending on the mammal's response to treatment.

In some embodiments, the methods provided herein can include monitoring a treated subject to determine whether or not the combination therapy is effective. For example, a mammal having a tumor (e.g., a human cancer patient) can be monitored to determine whether the tumor has decreased in size after treatment, or whether the number of tumor cells detected in the patient is reduced following treatment. In some cases, a post-treatment level of sPD-L1 can be measured in a biological sample taken from a mammal after treatment according to the methods described herein, and the level can be compared to a pre-treatment level of sPD-L1 in a corresponding sample taken from the mammal prior to treatment. A decrease in the level of sPD-L1 after treatment, as compared to the level in the sample taken before treatment or at an earlier time point during treatment, can indicate that the treatment is effective. An increase or no change in the level of sPD-L1 after treatment can indicate that the treatment is not effective.

The invention will be further described in the following examples, which do not limit the scope of the invention described in the claims.

### EXAMPLES

### Example 1—Effect of Selective Protease Inhibitors on sPD-L1 Production

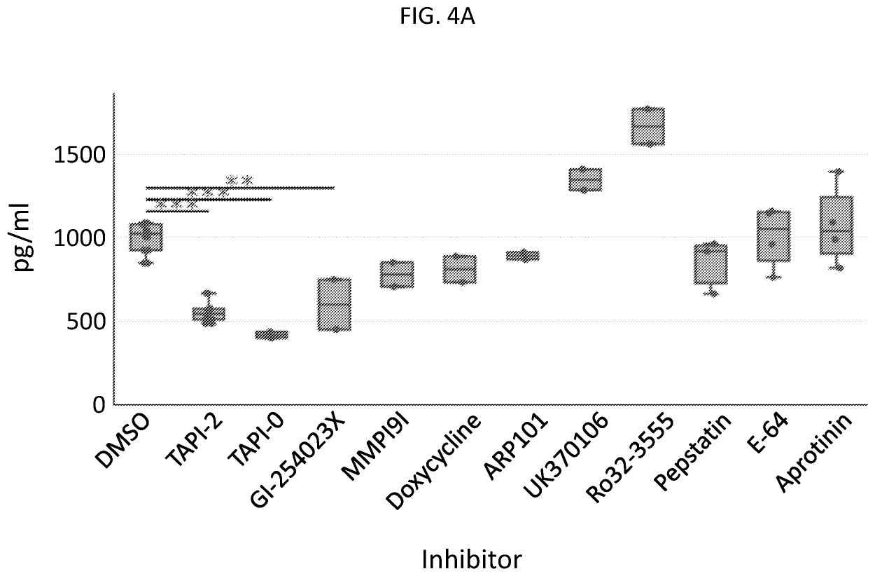

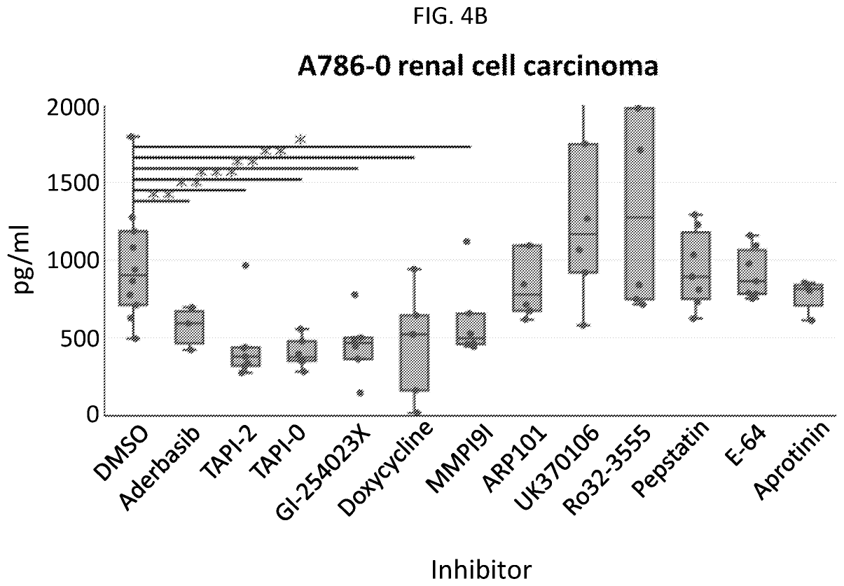

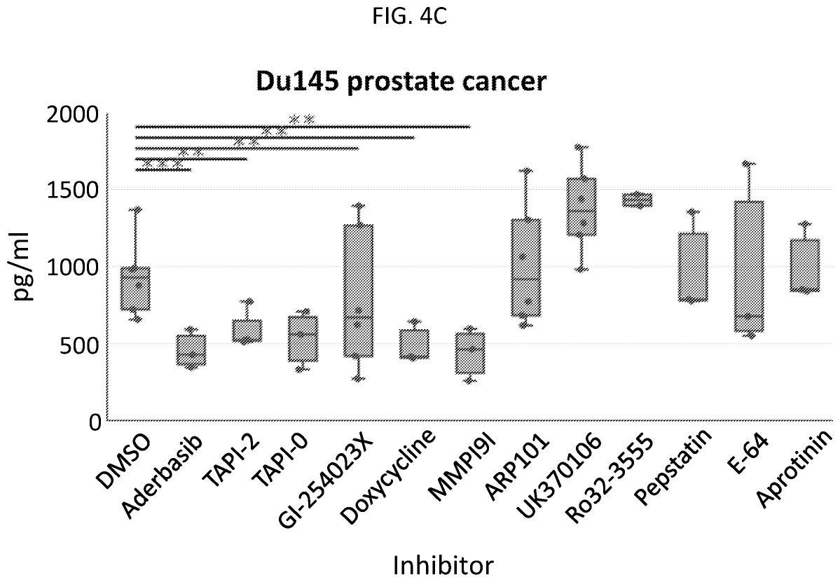

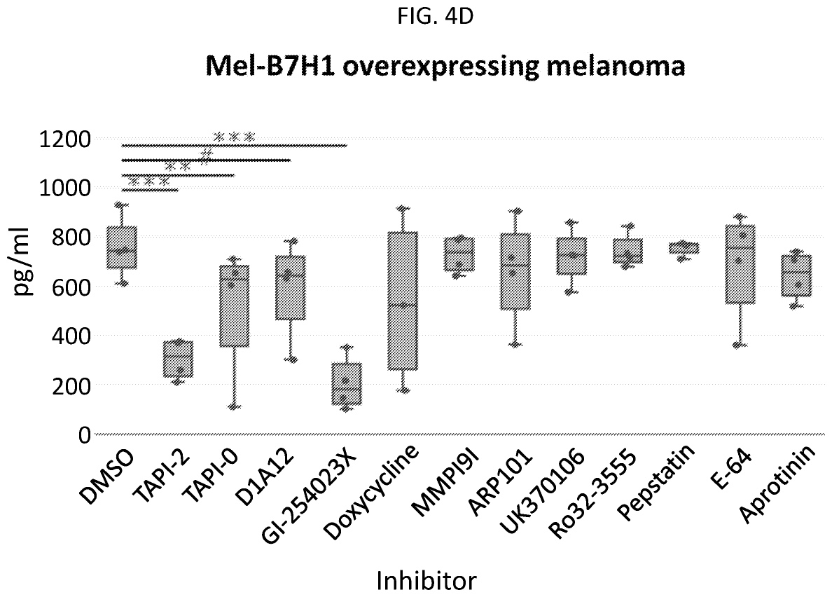

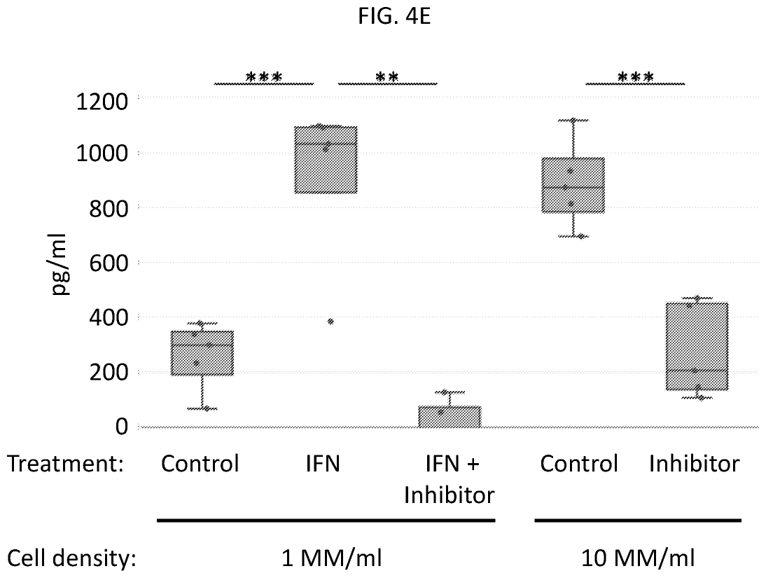

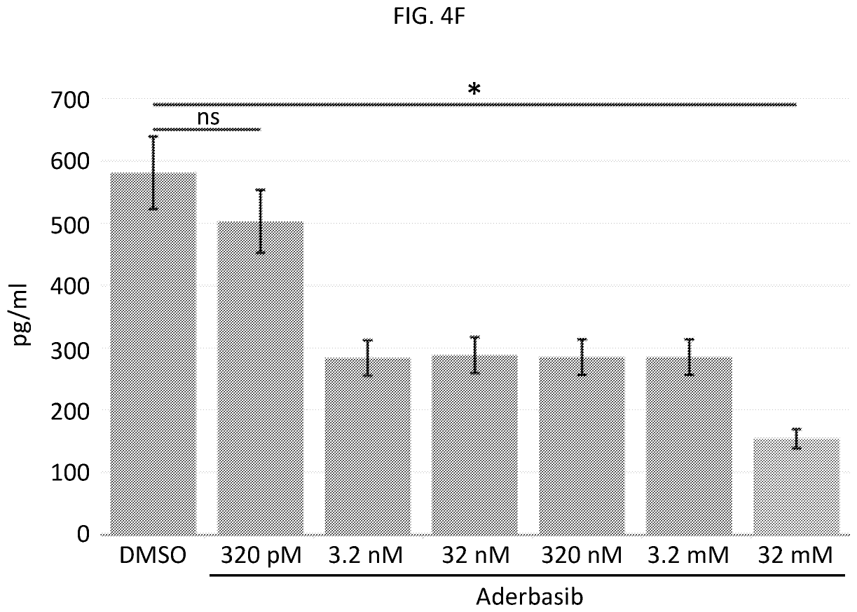

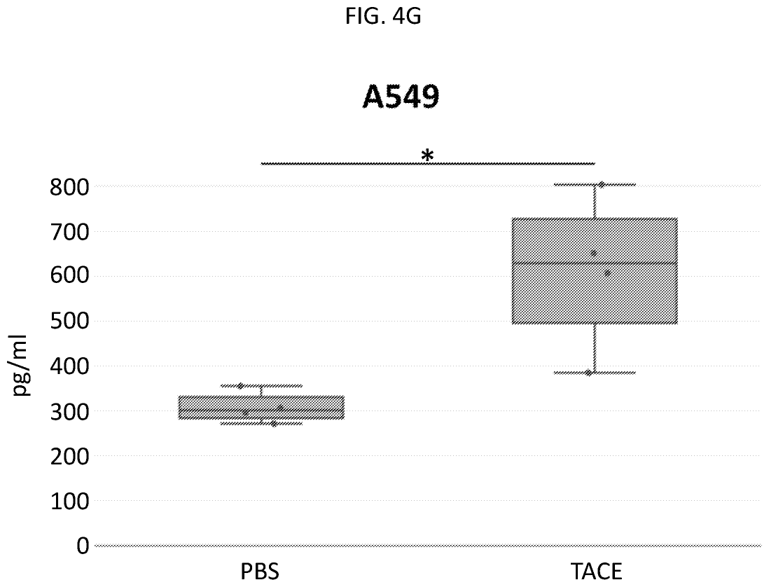

Several tumor cell lines were treated with PBS (placebo) or with various protease inhibitors, including matrix metalloprotease inhibitors TAPI-2 and TAPI-0, aspartic protease inhibitor Pepstatin, cysteine protease inhibitor E-64, serine protease inhibitor Aprotinin, combinations of these inhibitors, and an array of other matrix metalloprotease inhibitors. Cell supernatants were queried for sPD-L1 by ELISA as published elsewhere (Frigola et la., supra). In brief, paired mouse IgG2 monoclonal antibodies against extracellular rhB7-H1 were utilized in a capture-detection plate assay using biotinylation and HRP-streptavidin detection. This assay is specific for sPD-L1 and does not exhibit cross-reactivity to other B7-H homologues. Concentrations were determined by optical density measurements along a known standard curve of rhB7-H1. Karpas-299 cells were incubated at 10 MM cells per ml in cell culture media (RPMI plus FBS plus Penicillin/Streptomycin plus buffering reagents) over 24 hours in the presence of 1 μl/100 μl DMSO or PBS (placebo), TAPI-2 at 278 μM, Pepstatin at 206.5 μM, E-64 at 185 μM, or Aprotinin at 220 μM, or a combination of the above materials at various concentrations. These studies showed that the broad matrix metalloprotease inhibitor, TAPI-2, reduced the secretion of sPD-L1 (FIG. 4A; p=5.6×10⁻¹²) without reducing PD-L1 mRNA expression as determined by RT-PCR (not shown). All statistical analyses were performed using R! software. Two-sided, unpaired Student t tests assessed statistical differences in experimental groups except where otherwise indicated. P values indicate comparison to vehicle control groups. P<0.05 was considered statistically significant.

Subsequent studies were conducted in which Karpas-299 cells were incubated with different matrix metalloprotease inhibitors with varying specificities. For example, Karpas-299 cells were incubated at 10 MM cells per ml in cell culture media over 24 hours in the presence of 1 μl/100 μl DMSO (control), 50 μM TAPI-2, 50 μM TAPI-0, 50 μM GI254023X, 5 μM Ro 32-3555, 5 μM ARP 101, 50 μM UK 370106, 500 pM MMP-9 inhibitor I, 5 μM Doxycycline, or some combination thereof. TAPI-0, which is most active against ADAM17, reduced the secretion of sPD-L1 from Karpas-299 cells (FIG. 4A; p=3.97×10⁻⁷), without reducing PD-L1 mRNA expression (not shown).

Further studies were conducted using different types of cells. TAPI-2 significantly reduced sPD-L1 secretion from A786-0 (RCC) cells grown between 1 MM and 10 MM cells per ml (FIG. 4B; p=0.009, 0.00157, 0.00044, and 0.00156, respectively). Dul45 prostate cancer cells cultured with aderbasib, TAPI-2, TAPI-0, MMPI9, or doxycycline exhibited significantly reduced sPD-L1 secretion versus controls (FIG. 4C; p=0.00348, 0.0107, 0.0208, 0.0067, and 0.00569 respectively). sPD-L1 expression from Mel-B7H1 (transgenic melanoma) cells was most significantly reduced by TAPI-2 and GI254023X (FIG. 4D). Similar results were seen in cell lines induced to produce sPD-L1 by either interferon treatment or high cell titer (FIG. 4E).

Aderbasib is a selective inhibitor of matrix metalloproteases ADAM10 and ADAM17. Karpas-299 cells were incubated at 1 MM-10 MM cells/ml and treated for 24 hours with concentrations of aderbasib ranging from 320 pM to 32 mM. As indicated in FIG. 4F, Karpas-299 cells treated with increasing concentrations of aderbasib produced decreasing concentrations of sPD-L1.

In further studies, A549 cells, which do not produce large amounts of sPD-L1, were treated with control (PBS) or with a commercially-obtained, recombinant form of ADAM17 (TACE). A549 cells treated with exogenous ADAM17 (TACE) secreted higher amounts of detectable sPD-L1 than those treated with PBS control (FIG. 4G).

### Example 2—Effect of sPD-L1-Rich or Poor Supernatants on CD8+ T Cell Survival

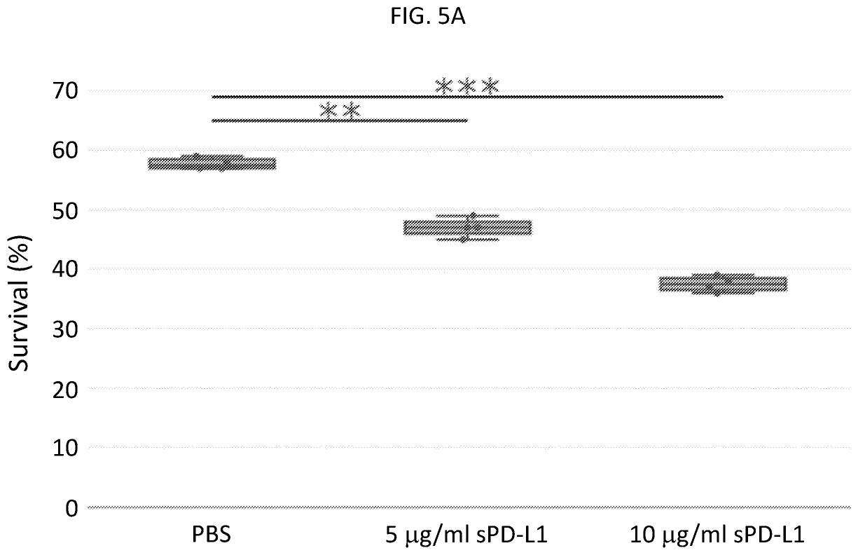

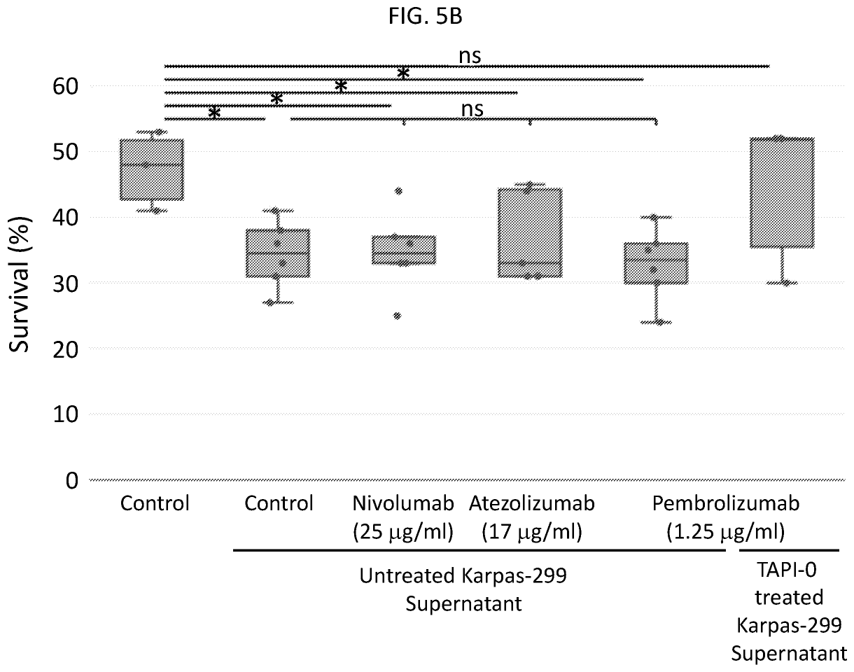

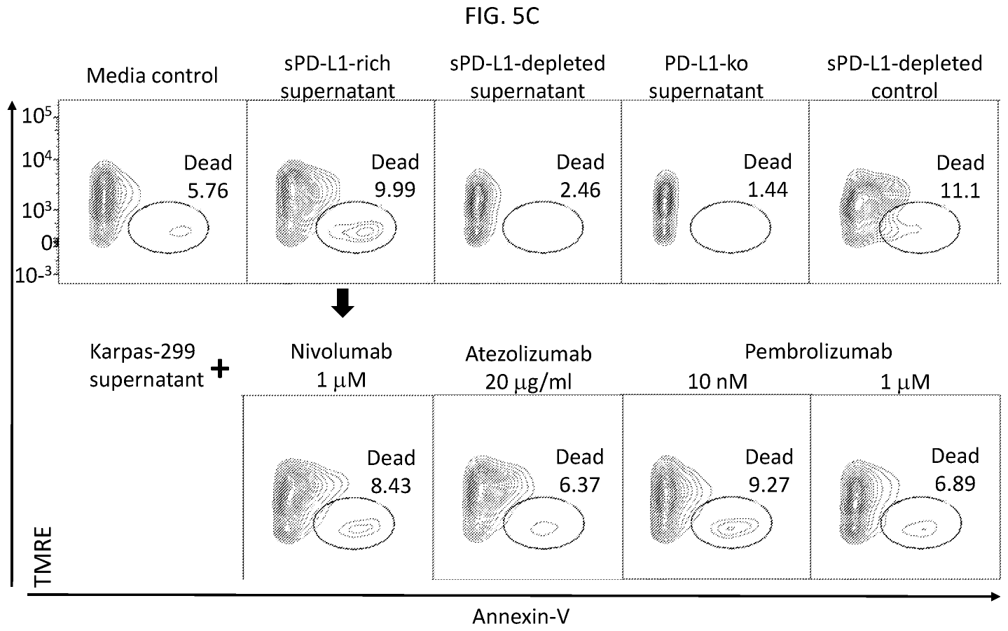

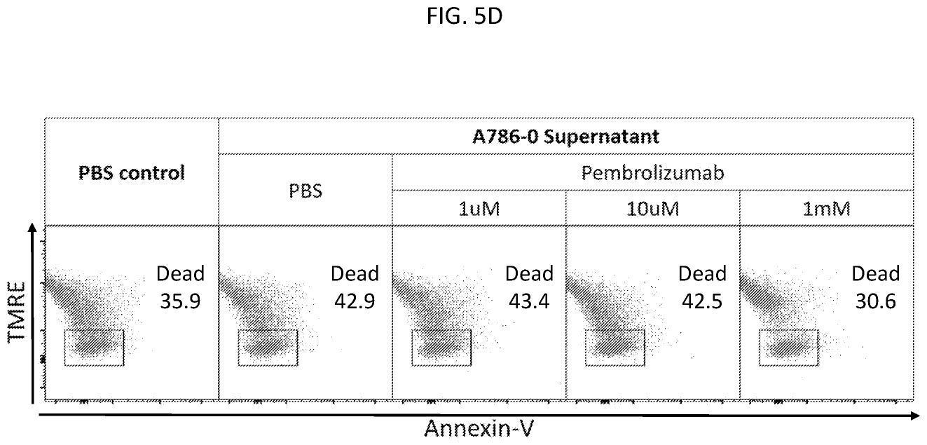

To determine whether sPD-L1 affects antitumor immunity, CD8+ T cells were isolated from healthy volunteers (see, Frigola et al., Clin Cancer Res, 1(177):1915-1923, 2011, for basic experimental protocols) and cultured with PBS or increasing concentrations of recombinant PD-L1. Survival was measured by trypan blue staining (FIG. 5A). CD8+ T cell survival was significantly reduced in cultures containing 5 μg/ml recombinant PD-L1 (p<0.0001) and 10 μg/ml recombinant PD-L1 (p<0.001).

Karpas-299 is a cancerous regulatory T cell line that expresses FoxP3 and exhibits immunosuppressive activity (Wolke et al., Int J Mol Med, 17(2): 275-278, 2006). Karpas-299 cells express PD-L1 that is cleaved into sPD-L1 by matrix metalloprotease ADAM17, and thus are PD-1 resistant. To determine whether tumor-derived sPD-L1 interferes with antitumor immune function, human CD8+ T cells were cultured with sPD-L1-rich Karpas-299 supernatant (FIG. 5B). Karpas-299 supernatant significantly decreased CD8+ T cell survival (p=0.005). Adding therapeutic doses of PD1 inhibitors nivolumab and pembrolizumab or PD-L1 checkpoint inhibitor atezolizumab did not rescue CD8+ T survival (p=0.011, 0.022, and 0.001, respectively).

CD8+ T cells isolated from healthy subjects were incubated in the presence of 50% Karpas-299 supernatants of varying types and 50% fresh cell culture media. Supernatant types included sPD-L1-rich supernatants from Karpas-299 cells, supernatants from Karpas-299 cells treated with TAPI-0 to prevent sPL-L1 production, supernatants from Karpas-299 cells “spiked” with TAPI-0 after sPD-L1 production, and fresh culture media controls (FIG. 5C). Karpas-299 cell supernatants were generated by incubation at 10 MM cells/ml for 48 hours. TMRE/Annexin-V binding was measured by flow cytometry to assess the level of CD8+ T cell survival. Antibodies and reagents used for flow cytometry were Annexin-V-FITC, anti-mouse CD16/32 (BioLegend; San Diego, Calif.); anti-PD-L1, mouse isotype control (eBioScience/Thermo Fisher Scientific; Waltham, Mass.), TMRE-PE (Abcam; Cambridge, Mass.), BD Wash/Perm (Becton Dickenson; Waltham, Mass.). Flow cytometry was performed and analyzed using FlowJo (Treestar; Ashland, Oreg.). Cells incubated in sPD-L1-rich Karpas-299 supernatants underwent apoptosis at a higher rate (˜10%) than those incubated in supernatants from TAPI-0 treated Karpas-299 cells (2.46%). Karpas-299 cell supernatants with added TAPI-0 did not induce cell death. Thus, sPD-L1-rich supernatants killed CD8+ T cells (previously shown to be part of tumor immunoevasion), whereas sPD-L1-poor supernatants did not.

Further studies showed that high doses of PD1 checkpoint inhibitors were able to overcome sPD-L1 by competitive inhibition. CD8+ T cells were incubated with supernatants from Karpas-299 cells as above, plus varying doses of several anti-PD-1 antibodies. Nivolumab (1 μM), atezolizumab (20 μg/ml), and pembrolizumab (10 nM and 1 μM) all increased the survival of CD8+ T cells to a certain extent (FIG. 5C), although the atezolizumab and high dose (1 μM) pembrolizumab were most effective.

Increasing concentrations of pembrolizumab also increased the survival of CD8+ T cells incubated with supernatants from A787-0 RCC cells (FIG. 5D). In brief, A786-0 cells were incubated at 10 MM cells/ml for 48 hours. Activated CD8+ T cells were incubated at 1 MM cells/ml in 80% RPMI and 20% A786-0 supernatant, plus varying doses of several anti-PD-1 antibodies. Increasing (and physiologically untenable) doses of pembrolizumab partially outcompeted sPD-L1 to partially rescue CD8 T cell survival.

It is to be noted that the foregoing examples illustrating the effects of sPD-L1 and inhibitors on CD8 T cells should not be considered as limiting the effects of combined PD1 therapy and MMP inhibition to only this subset of immune cells. Other cells, including but not limited to NK cells, macrophages, neutrophils, CD4 T cells, regulatory T cells, B cells, and others, also respond to sPD-L1 activity.

### Example 3—Soluble B7-H1 is Cleaved by ADAM10/17 from Tumors and Outcompetes Anti-PD1 Antibody to Induce Apoptosis of Activated CD8+ T Cells

Karpas-299 lymphoma cells producing sB7-H1 were cultured with an array of protease inhibitors. Supernatants from these cultures were isolated and analyzed for sB7H1 concentration by ELISA. Subsequently, activated human CD8+ T cells were cultured with recombinant sB7H1 protein and cell survival was measured at 24 hours. CD8+ T cells also were cultured with sB7H1-rich tumor cell line supernatants versus sB7H1-depleted supernatants over 48 hours in the presence of placebo, low dose (10 nM), or high dose (1 μM) anti-PD-1 antibody pembrolizumab, and cell survival was measured.

These studies revealed that the ADAM10 inhibitor GI254023X, the ADAM17 inhibitor TAPI-0, and the broad metalloprotease inhibitor TAPI-2 inhibited Karpas-299 production of sB7H1 (p<0.0001, respectively). Specific inhibitors of other metalloproteases did not significantly alter sB7H1 production. Exogenous sB7H1 at 5 μg/ml and 10 μg/ml induced cell death of CD8+ T cells (p<0.0001, respectively). Further, exogenous sB7H1-rich supernatants induced death of CD8+ T cells, whereas sB7H1-depleted supernatants did not. High-dose pembrolizumab decreased sB7-H1-containing supernatant-induced CD8+ T cell death. Thus, matrix metalloprotease inhibitors of ADAM10/17 mediate sB7H1 production in these lymphoma cells. sB7H1 induces apoptosis of activated CD8+ T cells, providing a possible explanation for PD-1 inhibitor resistance in various lymphomas and other cancers. In addition, sB7H1 may compete with anti-PD-1 antibody to engage PD-1 expressed by T cells and consequently reduce the therapeutic effects of anti-PD-1 antibody.

### Example 4—Primary Melanoma ADAM10 and ADAM17 Expression Correlates Negatively with PD-L1 Expression and Positively with sPD-L1 Expression

To determine whether tumors produce sPD-L1 in patients with melanoma, primary melanoma samples were obtained and stained for ADAM10, ADAM17, PD-1, and PD-L1 by IHC. Tissue blocks from human subjects were obtained as paraffin-embedded blocks. Specimens were sectioned (5 mm) and stained with antibodies against ADAM10 (Abcam), ADAM17 (Lifespan Biosciences; Seattle, Wash.), PD-1 (Abcam), and PD-L1 (Cell Signaling Technology, Inc.; Beverley, Mass.). Slides were counterstained with hematoxylin and fixed prior to imaging. Tumor cell lines were spun onto slides and fixed with acetone. These specimens were stained with antibodies against ADAM10 (Abcam), ADAM17 (Lifespan Biosciences), and PD-L1 (extracellular clone H1A).

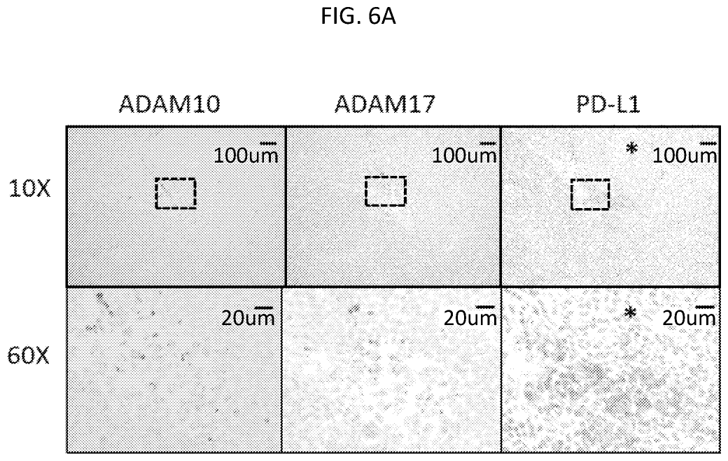

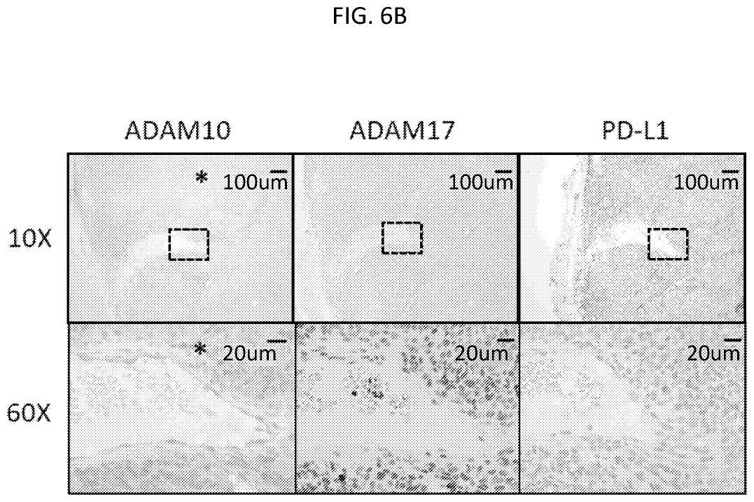

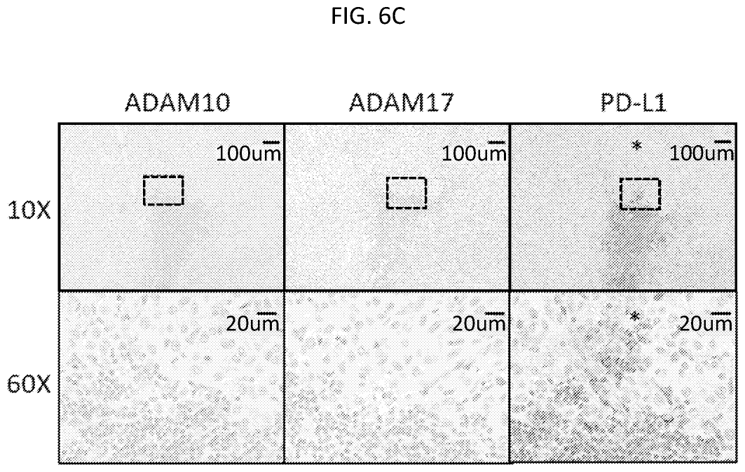

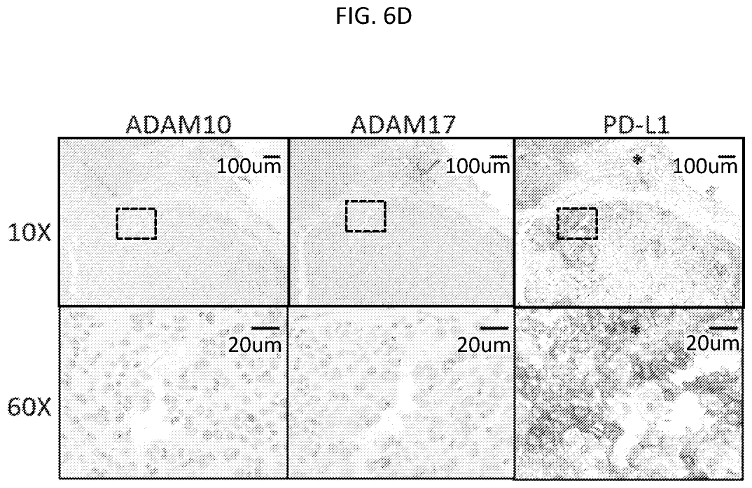

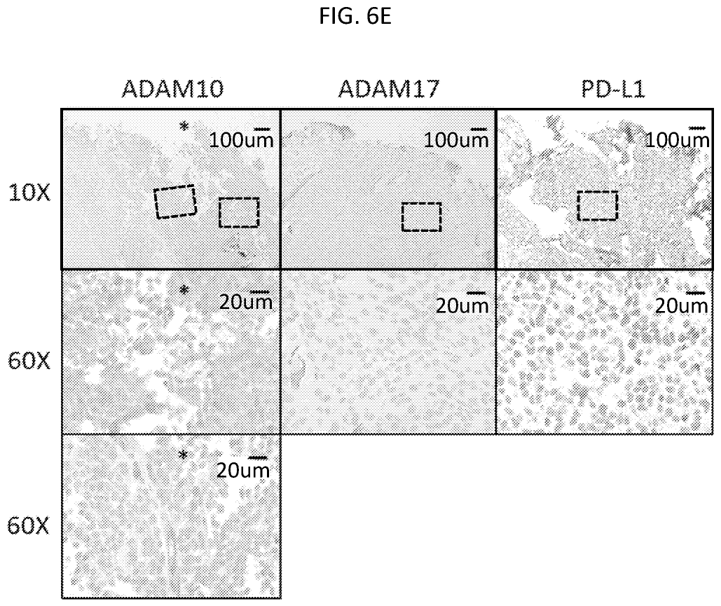

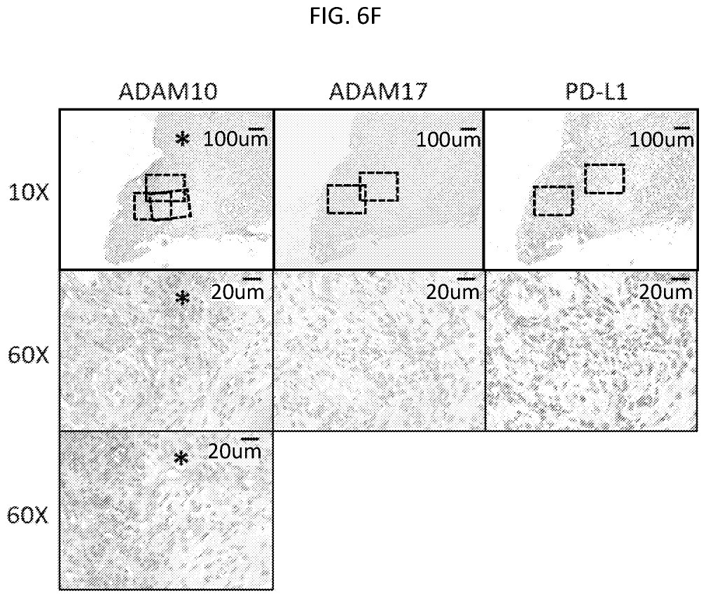

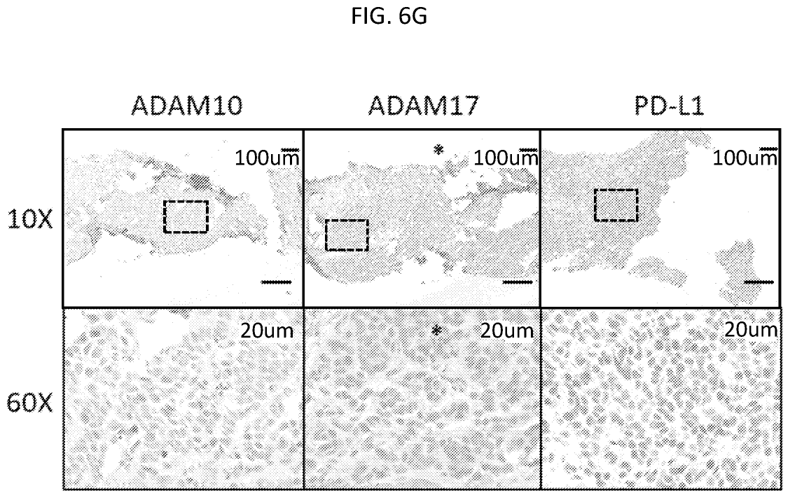

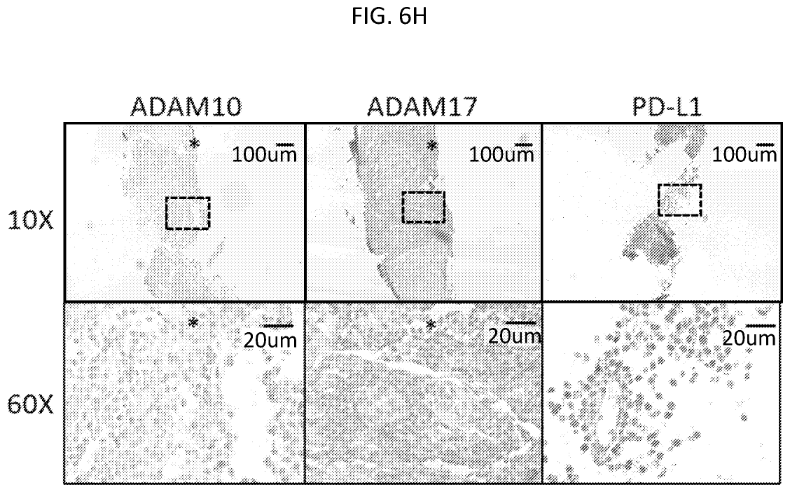

As shown in FIGS. 6A through 6H, melanoma tumor ADAM10 or ADAM17 positivity correlated with negative PD-L1 staining; conversely, tumor cell PD-L1 positivity correlated with negative ADAM10 and ADAM17 staining. Notably, variations in staining were seen in some tumor samples. In these samples, some locations stained positively for PD-L1 but not ADAM10 or ADAM17 while other locations stained negatively for PD-L1 but stained positively for either ADAM10 or ADAM17. Pearson chi squared independence test showed a significant negative correlation between ADAM10 or ADAM17 positivity and PD-L1 positivity (p=0.038). As a positive control, ADAM10 was observed in the endothelial cells surrounding blood vessels.

### Example 5—PD-L1 Expression is Reduced by ADAM10/ADAM17 and Restored by Inhibiting ADAM10/ADAM17

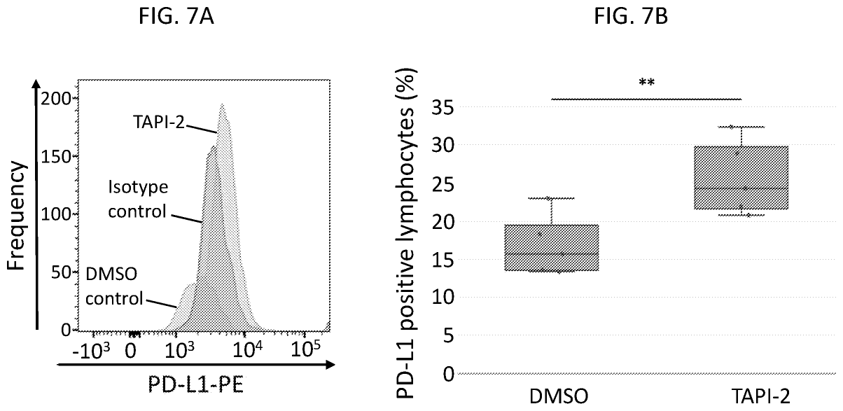

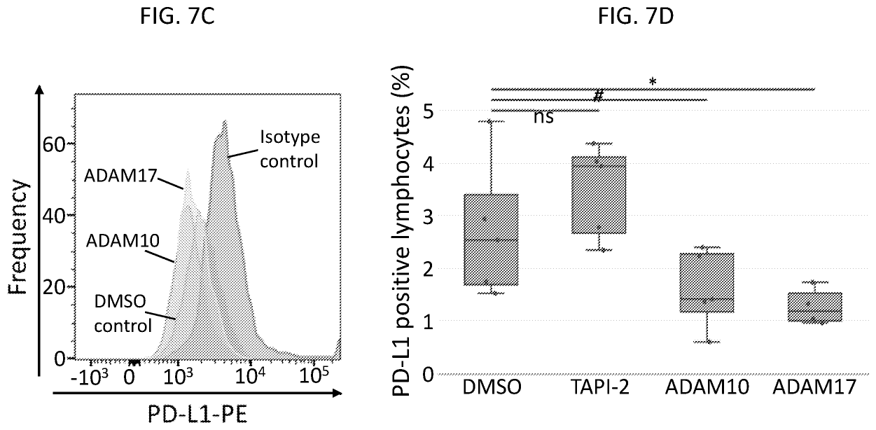

Karpas-299 cells, which shed soluble PD-L1, and A549 cells, which do not shed appreciable amounts of soluble PD-L1, were incubated in the presence of DMSO (control), TAPI-2, exogenous recombinant ADAM10, or ADAM17, and PD-L1 flow cytometry was performed. These studies demonstrated that PD-L1 expression was restored by treatment with the ADAM10/ADAM17 inhibitor, TAPI-2 (FIGS. 7A-7D), and was decreased by treatment with ADAM10 or ADAM17 (FIGS. 7C and 7D).

### Example 6—Kinetics of sPH-L1-Mediated Anti-PD-1 Resistance In Vitro

Supernatants from sPD-L1-secreting A786-0 cell lines are generated. Specifically, three distinct supernatants are produced: A786-0 sPD-L1-rich supernatant, ADAM10/17 inhibitor-treated A786-0 supernatant (lacking sPD-L1), and control A786-0 PD-L1 knockout cell supernatant (from a validated PD-L1 knockout A786-0 cell line). CD8+ T cells are isolated from healthy human donors (Frigola et al., supra). The CD8+ T cells are treated with sPD-L1-rich supernatants in the presence of escalating doses of the PD1 checkpoint therapeutics pembrolizumab (humanized anti-PD1), nivolumab (human anti-PD1), and atezolizumab (humanized anti-PD-L1), and TMRE/Annexin-V binding is measured by flow cytometry to ascertain the level of CD8+ T cell death. Calculated IC₅₀ values are compared to determine the relative concentration of each PD1 checkpoint therapeutic required to overcome apoptosis for each molar equivalent of sPD-L1 from the A786-0 supernatant.

In addition, PD-1-resistant melanoma (Mel624), NSCLC (A549), RCC (A7860), and non-Hodgkins lymphoma (Karpas-299) lines, as well as human CD8+ T cells are treated with escalating doses of sPD-L1 in the presence of pembrolizumab, and CD8+ T cell Bim expression, IL-2, production, and cell death are measured by flow cytometry, ELISA, or other methods known in the art. A kinetic profile is established for escalating doses of sPD-L1 vs. pembrolizumab interaction with PD-1 on the surface of the CD8+ T cells. Using sPD-L1-rich supernatants from Karpas-299 cells, supernatants from Karpas-299-PD-L1 knockout cells, and supernatants from Karpas-299 cells treated with an ADAM17 inhibitor to inhibit sPD-L1 production, a mechanistic model for tumor-mediated soluble immunotolerance factors is established.

### Example 7—Efficacy of Dual Anti-PD1 Anti-ADAM17 Therapy in Cancer

The data described herein suggest a novel approach of combined ADAM10/ADAM17 and PD-1 checkpoint inhibition for treatment of tumors. For example, a well-tolerated, orally bioavailable small molecule inhibitor of ADAM10/17 such as aderbasib may be used to prevent tumor production of sPD-L1, sensitizing the tumor to PD-1 checkpoint inhibition. Aderbasib reduces sPD-L1 production in malignant cell lines in a dose-dependent manner in vitro (FIG. 4F). Clinical trials are conducted to determine whether such a combination is tolerable and improves PD-1 checkpoint inhibitor therapy in patients with high serum sPD-L1 levels. Using ADAM17 inhibitors that have exhibited favorable toxicity profiles in Phase I and Phase II clinical trials (Infante et al., Breast Cancer Res Treat, 106:8269, 2007; Newton et al., J Clin Oncol, 28(15 Suppl.):3025-3025, 2010; Friedman et al., Cancer Res, 69(24 Suppl.):5056-5056, 2009; and Enniset al., J Cardiovasc Pharmacol Ther, 17(4):417-426, 2012), human subjects having NSCLC and exhibiting high serum sPD-L1 levels are treated with a PD-1 antagonist (e.g., pembrolizumab or nivolumab), with and without an orally bioavailable ADAM10/17 inhibitor (e.g., aderbasib), for 10 weeks. Baseline sPD-L1, radiographic studies, and Bim levels of CD11ahigh PD1+ CD8+ T cells are measured. Patients are randomized 1:1 to either pembrolizumab alone (standard therapy) or pembrolizumab plus ADAM10/17 inhibitor, and are followed at six-week intervals over the course of six months for survival, progression-free survival, peripheral T cell function, and serum sB7H1. Matched NSCLC cases with low sB7H1 also are followed as a control group with standard pembrolizumab therapy.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for enhancing effectiveness of an inhibitor of PD-1/PD-L1 interactions in a mammal identified as being in need thereof, said method comprising administering to said mammal (a) a metallopeptidase inhibitor and (b) an inhibitor of PD-1/PD-L1 interactions, wherein said metallopeptidase inhibitor is administered in an amount effective to reduce the level of soluble PD-1 ligand (sPD-L1) in the subject.

2. The method of claim 1, wherein said metallopeptidase inhibitor is a small molecule.

3. The method of claim 2, wherein said small molecule is aderbasib, XL784, KP-457, GI254023X, Ro 32-3555, ARP 101, UK 370106, MMP-9 inhibitor-I (MMP9I), doxycycline, TAPI-0, TAPI-1, or TAPI-2.

4. The method of claim 1, wherein said metallopeptidase inhibitor is an antibody.

5. The method of claim 4, wherein said antibody is MEDI3622 or D1(A12).

6. The method of claim 1, wherein said metallopeptidase inhibitor is an inhibitor of ADAM17.

7. The method of claim 6, wherein said inhibitor of ADAM17 is MEDI3622, D1(A12), aderbasib, XL784, KP-457, ADAM17 prodomain, TIMP-3, TAPI-0, TAPI-1, or TAPI-2.

8. The method of claim 1, wherein said metallopeptidase inhibitor is an inhibitor of ADAM10.

9. The method of claim 8, wherein said inhibitor of ADAM10 is aderbasib, GI254023X, ADAM10 prodomain, TIMP-1, or XL784.

10. The method of any one of claims 1 to 9, wherein said inhibitor of PD-1/PD-L1 interactions is an anti-PD-1 antibody.

11. The method of claim 10, wherein said anti-PD-1 antibody is pembrolizumab, nivolumab, or pidilizumab.

12. The method of any one of claims 1 to 9, wherein said inhibitor of PD-1/PD-L1 interactions is an anti-PD-L1 antibody.

13. The method of claim 12, wherein said anti-PD-L1 antibody is avelumab, atezolizumab, or durvalumab.

14. The method of any one of claims 1 to 13, wherein said mammal is a human.

15. The method of any one of claims 1 to 14, wherein said mammal is identified as being PD-1 resistant or PD-L1 resistant based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof.

16. The method of any one of claims 1 to 15, wherein said mammal is a cancer patient.

17. The method of any one of claims 1 to 16, wherein said mammal is identified as having a cancer selected from the group consisting of melanoma, non-small cell lung cancer (NSCLC), lymphoma, renal cell carcinoma (RCC), prostate cancer, bladder cancer, and colorectal cancer.

18. A method for immunomodulatory treatment, comprising administering to a mammal identified as being in need thereof (a) a metallopeptidase inhibitor and (b) an inhibitor of PD-1/PD-L1 interactions, wherein said metallopeptidase inhibitor is administered in an amount effective to reduce production of sPD-L1 in the subject, and wherein said inhibitor of PD-1/PD-L1 interactions is administered in an amount effective to modulate the activity of an immune cell within said mammal.

19. The method of claim 18, wherein said immune cell is a CD8 T-cell, a CD4 T-cell, a dendritic cell, a natural killer cell, a macrophage, or a stromal cell.

20. The method of claim 18 or claim 19, wherein said metallopeptidase inhibitor is a small molecule.

21. The method of claim 20, wherein said small molecule is aderbasib, XL784, KP-457, GI254023X, Ro 32-3555, ARP 101, UK 370106, MMP9I, doxycycline, TAPI-0, TAPI-1, or TAPI-2.

22. The method of claim 18 or claim 19, wherein said metallopeptidase inhibitor is an antibody.

23. The method of claim 22, wherein said antibody is MEDI3622 or D1(A12).

24. The method of claim 18 or claim 19, wherein said metallopeptidase inhibitor is an inhibitor of ADAM17.

25. The method of claim 24, wherein said inhibitor of ADAM17 is MEDI3622, D1(A12), aderbasib, XL784, KP-457, ADAM17 prodomain, TIMP-3, TAPI-0, TAPI-1, or TAPI-2.

26. The method of claim 18 or claim 19, wherein said metallopeptidase inhibitor is an inhibitor of ADAM10.

27. The method of claim 26, wherein said inhibitor of ADAM10 is aderbasib, GI254023X, ADAM10 prodomain, TIMP-1, or XL784.

28. The method of any one of claims 18 to 27, wherein said inhibitor of PD-1/PD-L1 interactions is an anti-PD-1 antibody.

29. The method of claim 28, wherein said anti-PD-1 antibody is pembrolizumab, nivolumab, or pidilizumab.

30. The method of any one of claims 18 to 27, wherein said inhibitor of PD-1/PD-L1 interactions is an anti-PD-L1 antibody.

31. The method of claim 30, wherein said anti-PD-L1 antibody is avelumab, atezolizumab, or durvalumab.

32. The method of any one of claims 18 to 31, wherein said mammal is a human.

33. The method of any one of claims 18 to 32, wherein said mammal is identified as being PD-1 resistant or PD-L1 resistant based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof.

34. The method of any one of claims 18 to 33, wherein said mammal is a cancer patient.

35. The method of any one of claims 18 to 34, wherein said mammal is identified as having a cancer selected from the group consisting of melanoma, NSCLC, lymphoma RCC, prostate cancer, bladder cancer, and colorectal cancer.

36. A method for reducing the number of cancer cells within a mammal, wherein the method comprises administering (a) a metallopeptidase inhibitor and (b) an inhibitor of PD-1/PD-L1 interactions to said mammal, wherein the number of cancer cells within said mammal are reduced.

37. The method of claim 36, wherein said cancer cells are melanoma cells, NSCLC cells, lymphoma cells, RCC cells, prostate cancer cells, bladder cancer cells, or colorectal cancer cells.

38. The method of claim 36 or claim 37, wherein said metallopeptidase inhibitor is a small molecule.

39. The method of claim 38, wherein said small molecule is aderbasib, XL784, KP-457, GI254023X, Ro 32-3555, ARP 101, UK 370106, MMP9I, doxycycline, TAPI-0, TAPI-1, or TAPI-2.

40. The method of claim 36 or claim 37, wherein said metallopeptidase inhibitor is an antibody.

41. The method of claim 40, wherein said antibody is MEDI3622 or D1(A12).

42. The method of claim 36 or claim 37, wherein said metallopeptidase inhibitor is an inhibitor of ADAM17.

43. The method of claim 42, wherein said inhibitor of ADAM17 is MEDI3622, D1(A12), aderbasib, XL784, KP-457, ADAM17 prodomain, TIMP-3, TAPI-0, TAPI-1, or TAPI-2.

44. The method of claim 36 or claim 37, wherein said metallopeptidase inhibitor is an inhibitor of ADAM10.

45. The method of claim 44, wherein said inhibitor of ADAM10 is aderbasib, GI254023X, ADAM10 prodomain, TIMP-1, or XL784.

46. The method of any one of claims 36 to 45, wherein said inhibitor of PD-1/PD-L1 interactions is an anti-PD-1 antibody.

47. The method of claim 46, wherein said anti-PD-1 antibody is pembrolizumab, nivolumab, or pidilizumab.

48. The method of any one of claims 36 to 45, wherein said inhibitor of PD-1/PD-L1 interactions is an anti-PD-L1 antibody.

49. The method of claim 48, wherein said anti-PD-L1 antibody is avelumab, atezolizumab, or durvalumab.

50. The method of any one of claims 36 to 49, wherein said method comprises administering two or more metallopeptidase inhibitors to said mammal.

51. The method of any one of claims 36 to 50, wherein said mammal is a human.

52. The method of any one of claims 36 to 51, wherein said mammal is identified as being PD-1 resistant or PD-L1 resistant based on elevated levels of ADAM10 in a tumor sample, elevated levels of ADAM17 in a tumor sample, elevated levels of sPD-L1 in a body fluid sample, reduced levels of PD-L1 in a tumor sample, or any combination thereof.

53. The method of any one of claims 36 to 52, wherein said mammal is a cancer patient.

54. The method of any one of claims 36 to 53, wherein said mammal is identified as having a cancer selected from the group consisting of melanoma, NSCLC, lymphoma, RCC, prostate cancer, bladder cancer, and colorectal cancer.

55. A method for treating a mammal identified as being resistant to an inhibitor of PD-1/PD-L1 interactions, said method comprising administering to said mammal (a) a metallopeptidase inhibitor and (b) an inhibitor of PD-1/PD-L1 interactions, wherein said metallopeptidase inhibitor is administered in an amount effective to reduce said resistance in said mammal.

56. The method of claim 55, wherein said metallopeptidase inhibitor is a small molecule.

57. The method of claim 56, wherein said small molecule is aderbasib, XL784, KP-457, GI254023X, Ro 32-3555, ARP 101, UK 370106, MMP9I, doxycycline, TAPI-0, TAPI-1, or TAPI-2.

58. The method of claim 55, wherein said metallopeptidase inhibitor is an antibody.

59. The method of claim 58, wherein said antibody is MEDI3622 or D1(A12).

60. The method of claim 55, wherein said metallopeptidase inhibitor is an inhibitor of ADAM17.

61. The method of claim 60, wherein said inhibitor of ADAM17 is MEDI3622, D1(A12), aderbasib, XL784, KP-457, ADAM17 prodomain, TIMP-3, TAPI-0, TAPI-1, or TAPI-2.

62. The method of claim 55, wherein said metallopeptidase inhibitor is an inhibitor of ADAM10.

63. The method of claim 62, wherein said inhibitor of ADAM10 is aderbasib, GI254023X, ADAM10 prodomain, TIMP-1, or XL784.

64. The method of any one of claims 55 to 63, wherein said inhibitor of PD-1/PD-L1 interactions is an anti-PD-1 antibody.

65. The method of claim 64, wherein said anti-PD-1 antibody is pembrolizumab, nivolumab, or pidilizumab.

66. The method of any one of claims 55 to 63, wherein said inhibitor of PD-1/PD-L1 interactions is an anti-PD-L1 antibody.

67. The method of claim 66, wherein said anti-PD-L1 antibody is avelumab, atezolizumab, or durvalumab.

68. The method of any one of claims 55 to 67, wherein said mammal is a human.

69. The method of any one of claims 55 to 68, wherein said mammal is identified as being anti-PD-1 resistant.

70. The method of any one of claims 55 to 68, wherein said mammal is identified as being anti-PD-L1 resistant.

71. The method of any one of claims 55 to 70, wherein said mammal is a cancer patient.

72. The method of any one of claims 55 to 71, wherein said mammal is identified as having a cancer selected from the group consisting of melanoma, NSCLC, lymphoma, RCC, prostate cancer, bladder cancer, and colorectal cancer.

73. A composition comprising a metallopeptidase inhibitor and an inhibitor of PD-1/PD-L1 interactions.

74. The composition of claim 73, further comprising a pharmaceutically acceptable carrier.

75. The composition of claim 74, wherein said pharmaceutically acceptable carrier comprises water, saline solution, a binding agent, a filler, a lubricant, a disintegrate, or a wetting agent.

76. The composition of any one of claims 73 to 75, wherein said metallopeptidase inhibitor is a small molecule.

77. The composition of claim 76, wherein said small molecule is aderbasib, XL784, KP-457, GI254023X, Ro 32-3555, ARP 101, UK 370106, MMP9I, doxycycline, TAPI-0, TAPI-1, or TAPI-2.

78. The composition of any one of claims 73 to 75, wherein said metallopeptidase inhibitor is an antibody.

79. The composition of claim 78, wherein said antibody is MEDI3622 or D1(A12).

80. The composition of any one of claims 73 to 75, wherein said metallopeptidase inhibitor is an inhibitor of ADAM17.

81. The composition of claim 80, wherein said inhibitor of ADAM17 is MEDI3622, D1(A12), aderbasib, XL784, KP-457, ADAM17 prodomain, TIMP-3, TAPI-0, TAPI-1, or TAPI-2.

82. The composition of any one of claims 73 to 75, wherein said metallopeptidase inhibitor is an inhibitor of ADAM10.

83. The composition of claim 82, wherein said inhibitor of ADAM10 is aderbasib, GI254023X, ADAM10 prodomain, TIMP-1, or XL784.

84. The composition of any one of claims 73 to 83, wherein said inhibitor of PD-1/PD-L1 interactions is an anti-PD-1 antibody.

85. The composition of claim 84, wherein said anti-PD-1 antibody is pembrolizumab, nivolumab, or pidilizumab.

86. The composition of any one of claims 73 to 83, wherein said inhibitor of PD-1/PD-L1 interactions is an anti-PD-L1 antibody.

87. The composition of claim 86, wherein said anti-PD-L1 antibody is avelumab, atezolizumab, or durvalumab.

88. A kit comprising a metallopeptidase inhibitor and an inhibitor of PD-1/PD-L1 interactions.

89. The kit of claim 88, wherein said metallopeptidase inhibitor is a small molecule.

90. The kit of claim 89, wherein said small molecule is aderbasib, XL784, KP-457, GI254023X, Ro 32-3555, ARP 101, UK 370106, MMP9I, doxycycline, TAPI-0, TAPI-1, or TAPI-2.

91. The kit of claim 88, wherein said metallopeptidase inhibitor is an antibody.

92. The kit of claim 91, wherein said antibody is MEDI3622 or D1(A12).

93. The kit of claim 88, wherein said metallopeptidase inhibitor is an inhibitor of ADAM17.

94. The kit of claim 93, wherein said inhibitor of ADAM17 is MEDI3622, D1(A12), aderbasib, XL784, KP-457, ADAM17 prodomain, TIMP-3, TAPI-0, TAPI-1, or TAPI-2.

95. The kit of claim 88, wherein said metallopeptidase inhibitor is an inhibitor of ADAM10.

96. The kit of claim 95, wherein said inhibitor of ADAM10 is aderbasib, GI254023X, ADAM10 prodomain, TIMP-1, or XL784.

97. The kit of any one of claims 88 to 96, wherein said inhibitor of PD-1/PD-L1 interactions is an anti-PD-1 antibody.

98. The kit of claim 97, wherein said anti-PD-1 antibody is pembrolizumab, nivolumab, or pidilizumab.

99. The kit of any one of claims 88 to 96, wherein said inhibitor of PD-1/PD-L1 interactions is an anti-PD-L1 antibody.

100. The kit of claim 99, wherein said anti-PD-L1 antibody is avelumab, atezolizumab, or durvalumab.

101. A system for immunomodulatory treatment, comprising a metallopeptidase inhibitor and an inhibitor of PD-1/PD-L1 interactions, wherein, when administered to a mammal with cancer, said metallopeptidase inhibitor and the inhibitor of PD-1/PD-L1 interactions are effective to increase immune system killing of cancer cells.

102. The system of claim 101, wherein said cancer cells are melanoma cells, NSCLC cells, lymphoma cells, RCC cells, prostate cancer cells, bladder cancer cells, or colorectal cancer cells.

103. The system of claim 101 or claim 102, wherein said metallopeptidase inhibitor is a small molecule.

104. The system of claim 103, wherein said small molecule is aderbasib, XL784, KP-457, GI254023X, Ro 32-3555, ARP 101, UK 370106, MMP9I, doxycycline, TAPI-0, TAPI-1, or TAPI-2.

105. The system of claim 101 or claim 102, wherein said metallopeptidase inhibitor is an antibody.

106. The system of claim 105, wherein said antibody is MEDI3622 or D1(A12).

107. The system of claim 101 or claim 102, wherein said metallopeptidase inhibitor is an inhibitor of ADAM17.

108. The system of claim 107, wherein said inhibitor of ADAM17 is MEDI3622, D1(A12), aderbasib, XL784, KP-457, ADAM17 prodomain, TIMP-3, TAPI-0, TAPI-1, or TAPI-2.

109. The system of claim 101 or claim 102, wherein said metallopeptidase inhibitor is an inhibitor of ADAM10.

110. The system of claim 109, wherein said inhibitor of ADAM10 is aderbasib, GI254023X, ADAM10 prodomain, TIMP-1, or XL784.

111. The system of any one of claims 101 to 110, wherein said inhibitor of PD-1/PD-L1 interactions is an anti-PD-1 antibody.

112. The system of claim 111, wherein said anti-PD-1 antibody is pembrolizumab, nivolumab, or pidilizumab.

113. The system of any one of claims 101 to 110, wherein said inhibitor of PD-1/PD-L1 interactions is an anti-PD-L1 antibody.

114. The system of claim 113, wherein said anti-PD-L1 antibody is avelumab, atezolizumab, or durvalumab.


SEQUENCE LISTING (11 sequences)

SEQ ID NO: 1 - protein, 748 aa, Homo sapiens
MVLLRVLILLLSWAAGMGGQYGNPLNKYIRHYEGLSYNVDSLHQKHQRAKRAVSHEDQFLRLDFHAHGRHFNLRMKRDTSLFSDEFKVETSNKVLDYDTSHIYTGHIYGEEGSFSHGSVIDGRFEGFIQTRGGTFYVEPAERYIKDRTLPFHSVIYHEDDINYPHKYGPQGGCADHSVFERMRKYQMTGVEEVTQIPQEEHAANGPELLRKKRTTSAEKNTCQLYIQTDHLFFKYYGTREAVIAQISSHVKAIDTIYQTTDFSGIRNISFMVKRIRINTTADEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPICGNGMVEQGEECDCGYSDQCKDECCFDANQPEGRKCKLKPGKQCSPSQGPCCTAQCAFKSKSEKCRDDSDCAREGICNGFTALCPASDPKPNFTDCNRHTQVCINGQCAGSICEKYGLEECTCASSDGKDDKELCHVCCMKKMDPSTCASTGSVQWSRHFSGRTITLQPGSPCNDFRGYCDVFMRCRLVDADGPLARLKKAIFSPELYENIAEWIVAHWWAVLLMGIALIMLMAGFIKICSVHTPSSNPKLPPPKPLPGTLKRRRPPQPIQQPQRQRPRESYQMGHMRR

SEQ ID NO: 2 - DNA, 2247 bp, Homo sapiens
atggtgttgctgagagtgttaattctgctcctctcctgggcggcggggatgggaggtcagtatgggaatcctttaaataaatatatcagacattatgaaggattatcttacaatgtggattcattacaccaaaaacaccagcgtgccaaaagagcagtctcacatgaagaccaatttttacgtctagatttccatgcccatggaagacatttcaacctacgaatgaagagggacacttcccttttcagtgatgaatttaaagtagaaacatcaaataaagtacttgattatgatacctctcatatttacactggacatatttatggtgaagaaggaagttttagccatgggtctgttattgatggaagatttgaaggattcatccagactcgtggtggcacattttatgttgagccagcagagagatatattaaagaccgaactctgccatttcactctgtcatttatcatgaagatgatattaactatccccataaatacggtcctcaggggggctgtgcagatcattcagtatttgaaagaatgaggaaataccagatgactggtgtagaggaagtaacacagatacctcaagaagaacatgctgctaatggtccagaacttctgaggaaaaaacgtacaacttcagctgaaaaaaatacttgtcagctttatattcagactgatcatttgttctttaaatattacggaacacgagaagctgtgattgcccagatatccagtcatgttaaagcgattgatacaatttaccagaccacagacttctccggaatccgtaacatcagtttcatggtgaaacgcataagaatcaatacaactgctgatgagaaggaccctacaaatcctttccgtttcccaaatattggtgtggagaagtttctggaattgaattctgagcagaatcatgatgactactgtttggcctatgtcttcacagaccgagattttgatgatggcgtacttggtctggcttgggttggagcaccttcaggaagctctggaggaatatgtgaaaaaagtaaactctattcagatggtaagaagaagtccttaaacactggaattattactgttcagaactatgggtctcatgtacctcccaaagtctctcacattacttttgctcacgaagttggacataactttggatccccacatgattctggaacagagtgcacaccaggagaatctaagaatttgggtcaaaaagaaaatggcaattacatcatgtatgcaagagcaacatctggggacaaacttaacaacaataaattctcactctgtagtattagaaatataagccaagttcttgagaagaagagaaacaactgttttgttgaatctggccaacctatttgtggaaatggaatggtagaacaaggtgaagaatgtgattgtggctatagtgaccagtgtaaagatgaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagactgtgcaagggaaggaatatgtaatggcttcacagctctctgcccagcatctgaccctaaaccaaacttcacagactgtaataggcatacacaagtgtgcattaatgggcaatgtgcaggttctatctgtgagaaatatggcttagaggagtgtacgtgtgccagttctgatggcaaagatgataaagaattatgccatgtatgctgtatgaagaaaatggacccatcaacttgtgccagtacagggtctgtgcagtggagtaggcacttcagtggtcgaaccatcaccctgcaacctggatccccttgcaacgattttagaggttactgtgatgttttcatgcggtgcagattagtagatgctgatggtcctctagctaggcttaaaaaagcaatttttagtccagagctctatgaaaacattgctgaatggattgtggctcattggtgggcagtattacttatgggaattgctctgatcatgctaatggctggatttattaagatatgcagtgttcatactccaagtagtaatccaaagttgcctcctcctaaaccacttccaggcactttaaagaggaggagacctccacagcccattcagcaaccccagcgtcagcggccccgagagagttatcaaatgggacacatgagacgctaa

SEQ ID NO: 3 - protein, 258 aa, Homo sapiens
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIHTDRAN

SEQ ID NO: 4 - DNA, 777 bp, Homo sapiens
atgaggcagtctctcctattcctgaccagcgtggttcctttcgtgctggcgccgcgacctccggatgacccgggcttcggcccccaccagagactcgagaagcttgattctttgctctcagactacgatattctctctttatctaatatccagcagcattcggtaagaaaaagagatctacagacttcaacacatgtagaaacactactaactttttcagctttgaaaaggcattttaaattatacctgacatcaagtactgaacgtttttcacaaaatttcaaggtcgtggtggtggatggtaaaaacgaaagcgagtacactgtaaaatggcaggacttcttcactggacacgtggttggtgagcctgactctagggttctagcccacataagagatgatgatgttataatcagaatcaacacagatggggccgaatataacatagagccactttggagatttgttaatgataccaaagacaaaagaatgttagtttataaatctgaagatatcaagaatgtttcacgtttgcagtctccaaaagtgtgtggttatttaaaagtggataatgaagagttgctcccaaaagggttagtagacagagaaccacctgaagagcttgttcatcgagtgaaaagaagagctgacccagatcccatgaagaacacgtgtaaattattggtggtagcagatcatcgcttctacagatacatgggcagaggggaagagagtacaactacaaattacttaatacacacagatagagctaattga

SEQ ID NO: 5 - protein, 824 aa, Homo sapiens
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVTRSEKAASFKLQRQNRVDSKETEC

SEQ ID NO: 6 - DNA, 2475 bp, Homo sapiens
atgaggcagtctctcctattcctgaccagcgtggttcctttcgtgctggcgccgcgacctccggatgacccgggcttcggcccccaccagagactcgagaagcttgattctttgctctcagactacgatattctctctttatctaatatccagcagcattcggtaagaaaaagagatctacagacttcaacacatgtagaaacactactaactttttcagctttgaaaaggcattttaaattatacctgacatcaagtactgaacgtttttcacaaaatttcaaggtcgtggtggtggatggtaaaaacgaaagcgagtacactgtaaaatggcaggacttcttcactggacacgtggttggtgagcctgactctagggttctagcccacataagagatgatgatgttataatcagaatcaacacagatggggccgaatataacatagagccactttggagatttgttaatgataccaaagacaaaagaatgttagtttataaatctgaagatatcaagaatgtttcacgtttgcagtctccaaaagtgtgtggttatttaaaagtggataatgaagagttgctcccaaaagggttagtagacagagaaccacctgaagagcttgttcatcgagtgaaaagaagagctgacccagatcccatgaagaacacgtgtaaattattggtggtagcagatcatcgcttctacagatacatgggcagaggggaagagagtacaactacaaattacttaatagagctaattgacagagttgatgacatctatcggaacacttcatgggataatgcaggttttaaaggctatggaatacagatagagcagattcgcattctcaagtctccacaagaggtaaaacctggtgaaaagcactacaacatggcaaaaagttacccaaatgaagaaaaggatgcttgggatgtgaagatgttgctagagcaatttagctttgatatagctgaggaagcatctaaagtttgcttggcacaccttttcacataccaagattttgatatgggaactcttggattagcttatgttggctctcccagagcaaacagccatggaggtgtttgtccaaaggcttattatagcccagttgggaagaaaaatatctatttgaatagtggtttgacgagcacaaagaattatggtaaaaccatccttacaaaggaagctgacctggttacaactcatgaattgggacataattttggagcagaacatgatccggatggtctagcagaatgtgccccgaatgaggaccagggagggaaatatgtcatgtatcccatagctgtgagtggcgatcacgagaacaataagatgttttcaaactgcagtaaacaatcaatctataagaccattgaaagtaaggcccaggagtgttttcaagaacgcagcaataaagtttgtgggaactcgagggtggatgaaggagaagagtgtgatcctggcatcatgtatctgaacaacgacacctgctgcaacagcgactgcacgttgaaggaaggtgtccagtgcagtgacaggaacagtccttgctgtaaaaactgtcagtttgagactgcccagaagaagtgccaggaggcgattaatgctacttgcaaaggcgtgtcctactgcacaggtaatagcagtgagtgcccgcctccaggaaatgctgaagatgacactgtttgcttggatcttggcaagtgtaaggatgggaaatgcatccctttctgcgagagggaacagcagctggagtcctgtgcatgtaatgaaactgacaactcctgcaaggtgtgctgcagggacctttctggccgctgtgtgccctatgtcgatgctgaacaaaagaacttatttttgaggaaaggaaagccctgtacagtaggattttgtgacatgaatggcaaatgtgagaaacgagtacaggatgtaattgaacgattttgggatttcattgaccagctgagcatcaatacttttggaaagtttttagcagacaacatcgttgggtctgtcctggttttctccttgatattttggattcctttcagcattcttgtccattgtgtggataagaaattggataaacagtatgaatctctgtctctgtttcaccccagtaacgtcgaaatgctgagcagcatggattctgcatcggttcgcattatcaaaccctttcctgcgccccagactccaggccgcctgcagcctgcccctgtgatcccttcggcgccagcagctccaaaactggaccaccagagaatggacaccatccaggaagaccccagcacagactcacatatggacgaggatgggtttgagaaggaccccttcccaaatagcagcacagctgccaagtcatttgaggatctcacggaccatccggtcaccagaagtgaaaaggctgcctcctttaaactgcagcgtcagaatcgtgttgacagcaaagaaacagagtgctaa

SEQ ID NO: 7 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 8 - DNA, 867 bp, Homo sapiens
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 9 - protein, 150 aa, Homo sapiens [sequence omitted]

SEQ ID NO: 10 - DNA, 450 bp, Homo sapiens
ccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtg

SEQ ID NO: 11 - protein, 6 aa, Artificial Sequence
  synthetic peptide
IEGRMD